(19) United States
(12) Patent Application Publication
(10) Pub. No.: US 2008/0319415 A1
(43) Pub. Date: Dec. 25, 2008

(54) Balloon Angioplasty Device with Distal Protection Capability

(76) Inventor: Leonid Shturman, Nyon (CH)

Correspondence Address: Carter DeLuca Farrell & Schmidt, 445 Broad Hollow Road, Suite 225, Melville, NY, 11747, US

(21) Appl. No.: 11/919,692

(22) PCT Filed: May 25, 2006
(86) PCT No.: PCT/IB2006/051672

§ 371 Date: Oct. 31, 2007

(30) Foreign Application Priority Data
May 26, 2005 (GB) 0510800.6

(51) Int. Cl. A61M 29/02

(52) U.S. Cl. 604/509; 604/98.01; 604/103.01

(57) ABSTRACT

A balloon-angioplasty device for dilating a. region of a vessel of a patient which has been at least partially occluded by the presence of a stenotic lesion in the vessel is disclosed. The device comprises an elongate catheter shaft having an inflatable element located around the catheter shaft proximal to its distal end, the inflatable element comprising a dilatation balloon and a membrane which extends in a longitudinal direction around the dilatation balloon and has at least one flushing fluid opening located at or near its distal end. At least a portion of the flushing fluid that has entered the vessel through the or each flushing fluid opening (s) in the membrane is forced to flow in a retrograde direction between the treated stenotic lesion and the membrane.

The invention relates to a balloon angioplasty device and, in particular, to a balloon angioplasty catheter which is capable of providing protection to vessels and organs distal to the treatment site by preventing migration of debris in a distal direction along the vessel away from the catheter and the treatment site. More specifically, it relates to a balloon angioplasty catheter for dilating a region of a blood vessel (e.g., coronary or peripheral artery) or other biological tubular structure, which has been at least partially occluded by the presence of atherosclerotic stenotic lesions formed in a vessel of a patient with the aim of reducing the effect that the presence of the stenotic lesion has on restricting flow of blood or other fluid through the vessel. The balloon angioplasty catheter of the invention may be particularly useful in restoring the patency of vessels that have been constricted by the presence of stenotic lesions which may develop over a long period of time in venous or arterial shunts (grafts) used during coronary bypass surgery.

Atherosclerosis, the clogging of arteries, is a leading cause of coronary heart disease. Blood flow through the peripheral arteries (e.g., carotid, femoral, renal, etc.), is similarly affected by the development of atherosclerotic stenotic lesions (narrowings). One existing method of removing or reducing the affect of stenotic narrowings in blood vessels is known as angioplasty. A typical coronary angioplasty procedure consists of accessing a peripheral artery (usually femoral) and then advancing a guide catheter to the ostium of the coronary artery. A long coronary guidewire is then advanced through the guide catheter and the distal end of the guidewire is maneuvered through the coronary artery to a point beyond the stenotic lesion. A balloon angioplasty catheter with a furled inflatable balloon at its distal end is then advanced over the guidewire until the furled balloon is positioned across the stenotic or occluded area. The balloon is then inflated to dilate the constricted area. Very often a stent, which is pre-mounted on the balloon, is deployed at the same time to help keep the artery open. The angioplasty balloon is then deflated and a controlled injection of radiopaque contrast material is made in order to confirm successful dilation of the stenotic area. The balloon angioplasty catheter is then removed and an ultrasonic catheter may then be advanced over the guidewire to confirm proper stent deployment. The guidewire is subsequently removed from the body at the end of the procedure.

A disadvantage with the procedure described above is that particles of the stenotic lesion may break away from the stenotic thickening in the wall of the vessel if the balloon ruptures the fibrous cap (cover) of the stenosis. These particles can migrate along the coronary artery distally and block very small vessels (often capillaries) of the heart muscle. The detrimental effect caused by the presence of particulate debris produced as a result of this procedure is of major concern to physicians who practice in this field. Clearly, the existence of particulate matter in the coronary blood stream is undesirable and can cause potentially life-threatening complications, especially if the particles exceed a certain size.

It is known from U.S. Pat. No. 6,129,739 to mount a long blood permeable sac immediately proximal to the most flexible distal end portion of the guidewire. Such “distal protection” guidewire is advanced across the stenotic lesion distally to a position at which both the most flexible distal portion of the guidewire and the blood permeable sac are located distal to the stenotic lesion. Once the guidewire is in position, operation of a deployment member causes the sac to expand within the vessel to filter blood passing through it and catch particulate debris entrained in the blood. Once dilation of the stenotic lesion using the conventional balloon angioplasty catheter is complete, the sac can be collapsed to trap particulate matter therein for removal of the debris from the patient together with the distal protection guidewire. However, it will be appreciated that this distal protection device has a complicated construction and its blood permeable sac may itself become blocked with debris during treatment preventing further blood flow. It also requires accurate positioning of the sac distal to the stenotic lesion if it is to fulfil its function and the sac is to be deployed so as to catch particulate matter migrating in a distal direction. Furthermore, as the expandable sac is mounted on the guidewire and has to be placed distal to the stenotic lesion, the overall length of that portion of the guidewire that has to extend distally beyond the stenotic lesion must be significantly longer than compared to the portion of a conventional guidewire that commonly extends distally beyond the stenotic lesion. Therefore, use of the above described “distal protection” guidewire for treatment of atherosclerotic lesions located in distal portions of the coronary vessels may become very limited. It will also be appreciated that only particles of debris exceeding a certain size may be captured within the sac and other particulate matter may pass through it. Such particulate matter may still be of a sufficient size to cause blockage of very small vessels such as capillaries.

The present invention seeks to overcome or substantially alleviate the problems associated with conventional distal protection devices such as the distal protection device of the type described above.

According to the invention, there is provided a balloon angioplasty device for dilating a region of a vessel of a patient which has been at least partially occluded by the presence of a stenotic lesion in the vessel, the device comprising an elongate catheter shaft having an inflatable element located around the catheter shaft proximal to its distal end, the inflatable element comprising a dilatation balloon and a membrane which extends in a longitudinal direction around the dilatation balloon and has at least one flushing fluid opening located at or near its distal end, at least the dilatation balloon being attached at its distal end to the catheter shaft, the balloon angioplasty device also including a balloon inflation lumen in communication with the dilatation balloon and an antegrade flushing fluid flow path, a distal portion of said flushing fluid flow path being located between the dilatation balloon and the membrane so that, when the angioplasty device has been advanced over a guidewire into the vessel to be treated and the inflatable element of the angioplasty device has been positioned across the stenotic lesion of the vessel, the dilatation balloon is inflated by supplying pressurised inflation medium through the balloon inflation lumen into the dilatation balloon to cause the inflation element to dilate the stenotic lesion in the vessel, the inflation of the dilatation balloon being followed by its partial deflation during which the membrane continues to be pressed against the treated stenotic lesion by the pressure of flushing fluid which is pumped along the antegrade flushing fluid flow path between the dilatation balloon and the membrane, said pressurised flushing fluid entering the treated vessel through the or each flushing fluid opening(s) located at or near the distal end of the membrane, the membrane being pressed against the treated stenotic lesion to prevent the release of debris therefrom until the pressure of fluid in the vessel distal to the inflatable element increases to a pressure at which at least a portion of the flushing fluid that has entered the vessel through the or each flushing fluid opening(s) in the membrane is forced to flow in a retrograde direction between the treated stenotic lesion and the membrane when the membrane moves away from the treated stenotic lesion thereby providing at least one retrograde fluid flow path for retrograde flowing flushing fluid which entrains debris released from the stenotic lesion for subsequent removal of the retrograde flowing flushing fluid and debris entrained in such retrograde flowing flushing fluid from the treated vessel.

In one embodiment of the invention, the antegrade flushing fluid flow path includes a flushing fluid supply lumen which extends in a longitudinal direction within the catheter shaft and communicates distally with the distal portion of the antegrade flushing fluid flow path located between the dilatation balloon and the membrane of the inflatable element.

The flushing fluid supply lumen which extends within the catheter shaft has at least one distal opening which is located along the catheter shaft distal to an area of a proximal attachment of the membrane to the catheter shaft and proximal to an inflatable portion of the dilatation balloon.

In the preferred embodiment of the invention, the or each distal opening(s) of the flushing fluid supply lumen is located along the catheter shaft proximal to a proximal end of the dilatation balloon.

In the preferred embodiment of the invention, the membrane of the inflatable element, when inflated by the dilatation balloon, has a maximum diameter section which extends in a longitudinal direction around a maximum diameter section of the dilatation balloon, the maximum diameter section of the membrane continues distally into a transitional segment of the membrane which becomes progressively smaller in diameter as it converges towards the catheter shaft and finally continues into a minimum diameter segment of the membrane which is connected to the catheter shaft, the transitional segment of the membrane including the or each flushing fluid opening(s) of the membrane.

In the most preferred embodiment of the invention, the maximum diameter section of the membrane both distally and proximally extends beyond corresponding distal and proximal ends of the maximum diameter section of the dilatation balloon.

In the preferred embodiment of the invention, both dilatation balloon and the membrane are designed to be able together to withstand a certain maximum predetermined pressure of the inflation medium within the dilatation balloon and at least the dilatation balloon is designed to rupture if the pressure within the balloon exceeds a certain predetermined burst pressure.

The balloon and membrane may be formed from the same or different materials and either or both of the balloon and membrane may themselves be formed from multiple layers to provide them with specific predetermined strength and sufficient lubricity.

In one embodiment of the invention, the balloon and membrane of the inflatable element may be integrally formed together as one unitary component.

In another embodiment of the invention, the membrane of the inflatable element may be formed from an outer layer of the dilatation balloon.

In one embodiment of the invention both the dilatation balloon and the membrane are made from substantially non-stretchable material, the substantially non-stretchable membrane and the dilatation balloon being attached to each other at locations spaced around the balloon so that the membrane moves away from the treated stenotic lesion by being pulled away from the treated stenotic lesion by the collapsing dilatation balloon in the region extending over at least one location of attachment between the dilatation balloon and the membrane.

The dilatation balloon and the substantially non-stretchable membrane may be attached to each other at said spaced locations around the balloon along at least two longitudinally extending join lines or strips. The or each join-line or strip may be continuous or discontinuous. In the preferred embodiment at least two such join lines or strips are spaced circumferentially around the balloon at approximately 180 degrees relative to each other so as to be positioned substantially diametrically opposite each other. In a modified preferred embodiment, three or more longitudinally extending join lines or strips attaching the membrane to the dilatation balloon may be spaced circumferentially around the balloon.

In the preferred embodiment of the invention, the antegrade fluid flow path between the dilatation balloon and the membrane is normally closed as the dilatation balloon and the membrane lie substantially in contact with each other in the absence of any substantial flow of flushing fluid along the antegrade flushing fluid flow path, the distal portion of said antegrade flushing fluid flow path located between the balloon and the membrane opening in response to pumping of flushing fluid along the antegrade flushing fluid flow path to enable said flushing fluid to flow between the dilatation balloon and the membrane and into the vessel through the or each opening(s) in the membrane. The distal portion of said antegrade fluid flow path located between the dilatation balloon and the membrane can therefore be regarded as being a potential fluid flow path until fluid is actually pumped through it.

In one embodiment of the invention, the device may comprise means for controlling the pressure of the balloon inflation medium supplied to the dilatation balloon through the balloon inflation lumen relative to the pressure of the flushing fluid pumped in an antegrade direction between the dilatation balloon and the membrane, said means controlling the relative pressures of the balloon inflation medium and the flushing fluid so that the pressure of the flushing fluid in the vessel distal to the inflatable element reaches a level at which a fluid pressure gradient is generated within the vessel between a region distal to the inflatable element and a region proximal to the inflatable element, thereby causing at least a portion of the flushing fluid entering the vessel through the or each opening in the membrane to flow in a retrograde direction between the treated stenotic lesion and the membrane when the membrane moves away from the treated stenotic lesion.

In one embodiment of the invention, the device may include means for controlling the pressure of the balloon inflation medium supplied to the dilatation balloon through the balloon inflation lumen relative to the pressure of the flushing fluid pumped in an antegrade direction between the dilatation balloon and the membrane, said means controlling the relative pressures of the balloon inflation medium and the flushing fluid so that the pressure of the flushing fluid in the vessel distal to the dilatation balloon reaches a level at which a fluid pressure gradient is generated within the vessel between a region distal to the dilatation balloon and a region proximal to the dilatation balloon, thereby causing at least a portion of the flushing fluid entering the vessel through the or each opening in the membrane to flow in a retrograde direction between the treated stenotic lesion and the membrane when the membrane moves away from the treated stenotic lesion.

In yet another embodiment of the invention, the device may include means for controlling the relative pressure of the balloon inflation medium supplied to the dilatation balloon through the balloon inflation lumen relative to the pressure of the flushing fluid pumped in an antegrade direction between the dilatation balloon and the membrane, said means controlling the relative pressures of the balloon inflation medium and flushing fluid so that the pressure of the flushing fluid in the treated vessel reaches a level at which a fluid pressure gradient is generated within the vessel between a region distal to the point of entry of flushing fluid into the vessel and a region proximal to the point of entry of flushing fluid into the vessel, thereby causing at least a portion of the flushing fluid entering the vessel through the or each opening in the membrane to flow in a retrograde direction between the treated stenotic lesion and the membrane when the membrane moves away from the treated stenotic lesion.

In the preferred embodiment of the invention, the device may comprise means for controlling the pressure of the inflation fluid supplied to the dilatation balloon through the balloon inflation lumen relative to the pressure of the flushing fluid pumped in an antegrade direction between the dilatation balloon and the membrane, said means controlling these two pressures so that the membrane moves away from the treated stenotic lesion only after enough flushing fluid has entered the vessel through the or each opening in the membrane to cause at least a portion of the flushing fluid to flow in a retrograde direction along the fluid flow path formed between the treated stenotic lesion and the membrane when the membrane moves away from the treated stenotic lesion.

The device according to one embodiment of the invention includes means for generating a retrograde flow of flushing fluid entering the vessel through the or each opening in the membrane so that at least a portion of said flushing fluid flows along the retrograde fluid flow path between the membrane and the treated stenotic lesion.

In the preferred embodiment, the means for generating a retrograde flow of fluid along the flow path between the membrane and the treated stenotic lesion comprises a fluid pump operable to pump flushing fluid along the antegrade flushing fluid flow path into the vessel so that the pressure of fluid in the vessel distal to the dilatation balloon increases to a pressure at which at least a portion of the flushing fluid entering the vessel through the or each opening(s) in the membrane is forced to flow in a retrograde direction along the flow path between the treated stenotic lesion and the membrane when the membrane moves away from the treated stenotic lesion.

In one embodiment, the device may include a drainage sheath which extends in a longitudinal direction around the catheter shaft so that fluid flowing in a retrograde direction along the flow path between the membrane and the treated stenotic lesion flows into a lumen in the drainage sheath for removal of debris entrained in the retrograde flowing flushing fluid from the treated vessel.

In another embodiment, the device may include a drainage sheath which extends in a longitudinal direction around the catheter shaft so that fluid flowing in a retrograde direction along the flow path between the membrane and the treated stenotic lesion flows into a lumen in the drainage sheath for removal of debris entrained in the retrograde flowing flushing fluid from the patient.

The lumen in the drainage sheath is preferably defined by an annular space between the catheter shaft and the drainage sheath.

In a preferred embodiment the drainage sheath is longitudinally movable along the catheter shaft.

The means for generating, or assisting in the generation of, a retrograde flow of flushing fluid along the fluid flow path between the treated stenotic lesion and the membrane of the inflatable element may, in one embodiment, include a suction device in communication with the lumen in the drainage sheath operable to cause at least a portion of the flushing fluid entering the vessel to flow in a retrograde direction along said fluid flow path between the treated stenotic lesion and the membrane of the inflatable element and into the lumen in the drainage sheath together with debris released from the stenotic lesion for removal of said debris from the patient.

In one embodiment of the invention the suction device may include a peristaltic pump.

An inflatable cuff may be mounted on the drainage sheath at or near its distal end which, when inflated, restricts or blocks the antegrade flow of blood and retrograde flow of flushing fluid and debris between the inflatable cuff and the vessel wall as well as locates the drainage sheath in the vessel to be treated depending on the degree of inflation of the inflatable cuff.

The drainage sheath may include a cuff inflation lumen extending therethrough, the cuff inflation lumen communicating the inflatable cuff with an inflation source which is connected to the cuff inflation lumen at or close to the proximal end of the drainage sheath.

The inflatable cuff is preferably a balloon located adjacent to the distal end of the drainage sheath.

In a modified embodiment, the inflatable cuff comprises a stretchable sleeve extending around the drainage sheath and defining a normally closed but potentially inflatable space around the sheath, the stretchable sleeve having distal and proximal ends, the distal end of the sleeve being bonded to an outer surface of the drainage sheath near its distal end, the potentially inflatable space being in communication with an inflation device at or close to a proximal end of the sleeve so that the potentially inflatable space between the drainage sheath and the stretchable sleeve may be inflated to restrict or block antegrade flow of blood and retrograde flow of flushing fluid and debris between the stretchable sleeve and the vessel wall as well as locate the drainage sheath within the vessel to be treated, depending on the degree of inflation of the stretchable sleeve.

The catheter shaft may, in one embodiment, comprise a guidewire lumen which extends along the entire length of the shaft. In another embodiment, the guidewire lumen may extend proximally from the distal end of the shaft along only a portion of the shaft.

Any conventional angioplasty procedure includes placement of a guidewire across the stenotic lesion to be treated prior to advancement of the catheter shaft over the guidewire. Therefore, regardless of its length, the guidewire lumen in the angioplasty device of the invention is preferably designed to receive both conventional guidewires or guidewires designed for use in specific applications (e.g. “Rotablator” guidewire).

According to the invention, there is also provided a method of using a balloon angioplasty device for dilating a region of a vessel of a patient which has been at least partially occluded by the presence of a stenotic lesion in the vessel, the device comprising an elongate catheter shaft having an inflatable element located around the catheter shaft proximal to its distal end, the inflatable element comprising a dilatation balloon and a membrane which extends in a longitudinal direction around the dilatation balloon and has at least one flushing fluid opening located at or near its distal end, at least the dilatation balloon being attached at its distal end to the catheter shaft, the balloon angioplasty device also including a balloon inflation lumen in communication with the dilatation balloon and an antegrade flushing fluid flow path, a distal portion of said flushing fluid flow path being located between the dilatation balloon and the membrane, the method comprising the steps of:

(a) positioning the inflatable element of the angioplasty device across the stenotic lesion by advancing the angioplasty device over a guidewire into a vessel to be treated, the guidewire being placed in the vessel prior to being received in a guidewire lumen of the angioplasty device;
(b) inflating the dilatation balloon by pumping inflation medium through the balloon inflation lumen into the dilatation balloon to cause the inflatable element to dilate the vessel;
(c) partially deflating the dilatation balloon and maintaining the membrane pressed against the treated stenotic lesion to prevent release of debris therefrom by pumping flushing fluid through the antegrade flushing fluid flow path so that it flows through the distal portion of said flow path located between the dilatation balloon and the membrane and enters the treated vessel through the or each flushing fluid opening(s) in the membrane located at or near the distal end of the membrane;
(d) further deflating the dilatation balloon so that the membrane moves away from the treated stenotic lesion to form a retrograde fluid flow path between the treated stenotic lesion and the membrane for the retrograde flow of at least a portion of the flushing fluid that has entered the vessel through the or each flushing fluid opening(s) in the membrane to entrain debris released from the stenotic lesion in the retrograde flow of flushing fluid for subsequent removal of said flushing fluid and debris entrained in the flushing fluid from the treated vessel.

In the above described method the membrane, which extends over the dilatation balloon, is made from a non-stretchable material and is attached to the balloon at least two locations spaced around the balloon so that, during step (d) of further deflating the dilatation balloon, the balloon is deflated to a pressure at which the collapsing balloon pulls the non-stretchable membrane away from the treated stenotic lesion in the region extending over at least one location of attachment between the dilatation balloon and the membrane to form a retrograde fluid flow path between the membrane and the treated stenotic lesion for the retrograde flow of at least a portion of the flushing fluid that has entered the vessel through the or each flushing fluid opening(s) in the membrane to entrain debris released from the stenotic lesion in the retrograde flow of flushing fluid for subsequent removal of said flushing fluid and debris entrained in the flushing fluid from the treated vessel.

If the membrane of the inflatable element is made from a stretchable material, then during step (d) formation of the retrograde fluid flow path between the membrane and the treated stenotic lesion is achieved by combining the collapse of the dilatation balloon with a reduction of flushing fluid pressure to a level at which pressure of flushing fluid on the membrane is insufficient to press the membrane against the treated stenotic lesion and the stretchable material is allowed to partially regain its unstretched state to an extent at which the membrane is spaced away from both the stenotic lesion and the dilatation balloon thereby both preserving the antegrade fluid flow path for pumping flushing fluid between the balloon and the membrane and forming a retrograde fluid flow path between the membrane and the treated stenotic lesion.

In the above described method, during step (b) of inflating the dilatation balloon to dilate the stenotic lesion in the vessel, the distal portion of the antegrade flushing fluid flow path located between the dilatation balloon and the membrane is closed by the inflated dilatation which is pressed against the membrane, and the following step (c) of pumping flushing fluid along the antegrade flushing fluid flow path includes the step of using the pressure of the flushing fluid to open said distal portion of the antegrade flushing fluid flow path so that the flushing fluid can flow between the dilatation balloon and the membrane into the vessel through the or each flushing fluid opening(s) in the membrane.

In the above described method, the step (b) of supplying pressurised inflation medium into the dilatation balloon and the following step (c) of pumping flushing fluid along the antegrade flushing fluid flow path and into the vessel should include the step of controlling the pressure of the inflation medium supplied to the dilatation balloon relative to the pressure of the flushing fluid pumped into the vessel via the distal portion of the antegrade fluid flow path located between the membrane and the dilatation balloon, so that the membrane is pressed against the treated stenotic lesion by the pressure of the flushing fluid until the pressure of the flushing fluid in the vessel distal to the dilatation balloon is sufficient to cause at least a portion of the flushing fluid to flow along the retrograde fluid flow path between the membrane and the treated stenotic lesion when the membrane moves away from the treated stenotic lesion.

In the above described method, the step (c) of pumping flushing fluid along the antegrade flushing fluid flow path should comprise pumping flushing fluid at such volume rate that the pressure of the flushing fluid in the vessel distal to the inflatable element increases to a pressure that generates a fluid pressure gradient within the vessel between a region distal to the inflatable element and a region proximal to the inflatable element, thereby causing at least a portion of the flushing fluid to flow along said retrograde fluid flow path which is formed when the membrane moves away from the treated stenotic lesion.

In the above described method, the step (c) of pumping flushing fluid along the antegrade flushing fluid flow path comprises pumping flushing fluid at such volume rate that the pressure of the flushing fluid in the vessel distal to the dilatation balloon increases to a pressure that generates a fluid pressure gradient within the vessel between a region distal to the dilatation balloon and a region proximal to the dilatation balloon, thereby causing at least a portion of the flushing fluid to flow along said retrograde fluid flow path which is formed when the membrane moves away from the treated stenotic lesion.

In the above described method the (c) of pumping flushing fluid along the antegrade flushing fluid flow path comprises pumping flushing fluid at such volume rate that the pressure of the flushing fluid in the vessel distal to the inflatable element increases to a pressure that generates a fluid pressure gradient within the vessel between a region distal to the point of entry of flushing fluid into the vessel and a region proximal to the point of entry of flushing fluid into the vessel, thereby causing at least a portion of the flushing fluid to flow along said retrograde fluid flow path which is formed when the membrane moves away from the treated stenotic lesion.

Method step (b) of inflating the balloon to dilate the vessel and subsequent steps may be repeated several times. A period of time may be allowed to lapse between any one inflation of the dilatation balloon which is followed by subsequent steps leading to the retrograde flow of flushing fluid and the next inflation of the dilatation balloon, so that oxygenated blood can flow between the membrane and the vessel wall in an antegrade direction during this rest time period to maintain adequate supply of blood along the treated vessel to areas distal to the treatment site.

If the angioplasty device includes a drainage sheath which extends over the catheter shaft and is longitudinally movable with respect to the shaft, then the method preferably includes the step of moving the drainage sheath to a position at which its distal end is located proximal to the dilatation balloon so that flushing fluid and debris flowing along the retrograde fluid flow path between the membrane and the treated stenotic lesion flows into a lumen in the sheath for removal thereof from the patient.

In the embodiment in which an inflatable cuff is mounted on the drainage sheath, the method includes the step of inflating the inflatable cuff before initiating a flow of flushing fluid along the flushing fluid supply lumen to prevent antegrade flow of blood or retrograde flow of fluid and debris between the inflatable cuff of drainage sheath and the treated vessel.

If the device includes suction means in communication with the lumen in the sheath, the method may include the step of operating the suction means to draw flushing fluid and debris along the retrograde fluid flow path between the membrane and the treated stenotic lesion and into the lumen in the drainage sheath for subsequent removal from the patient.

Embodiments of the invention will now be described, by way of example only, with reference to the accompanying drawings, in which:

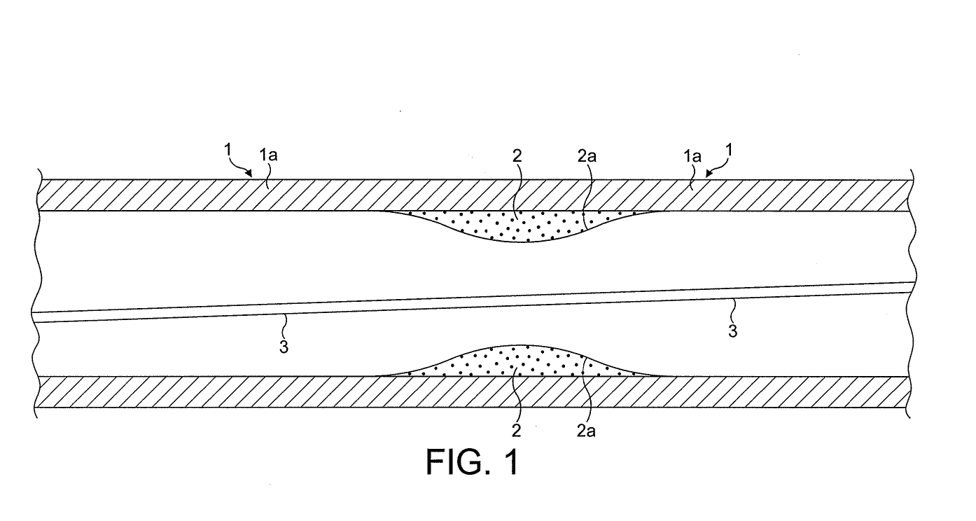

FIG. 1 is a sectional side elevation of a portion of a blood vessel which has been partially occluded by the presence of a stenotic lesion and in which a guidewire has been placed so that it extends across the stenotic lesion;

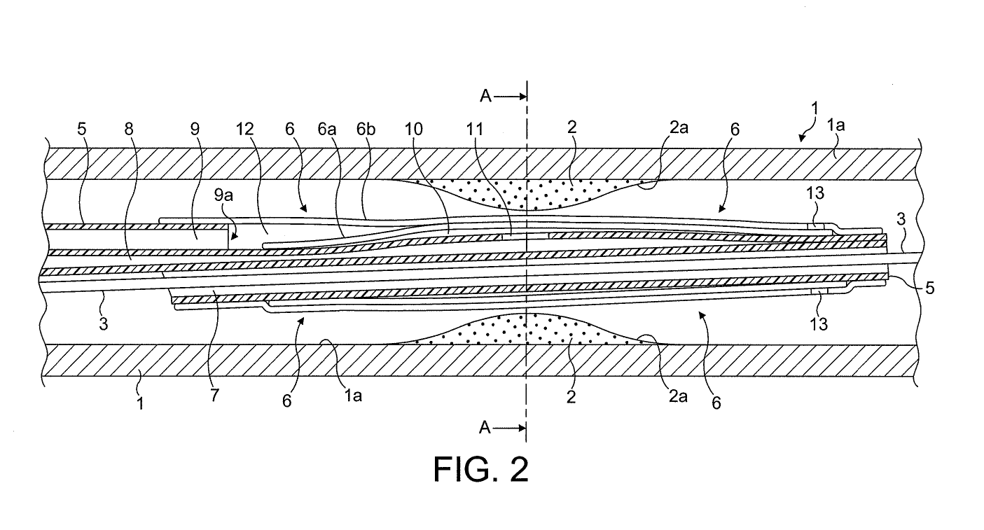

FIG. 2 is a side sectional view of the portion of the blood vessel shown in FIG. 1 which illustrates a side sectional view of a balloon angioplasty device of the invention which has been advanced over the guidewire until an inflatable element of the device comprising an elongated dilatation balloon and a membrane extending around the balloon, is disposed across the stenotic lesion of the vessel to be treated, the inflatable element being mounted on a distal end section of a catheter shaft of the device;

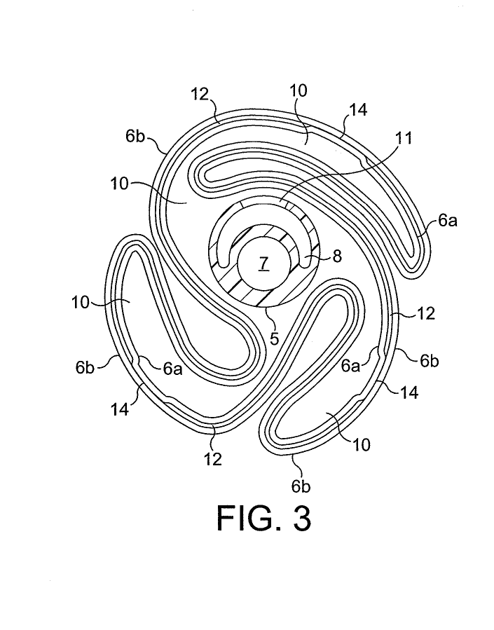

FIG. 3 is an enlarged cross-sectional view taken along the line A-A of FIG. 2 with the blood vessel and guidewire omitted for clarity;

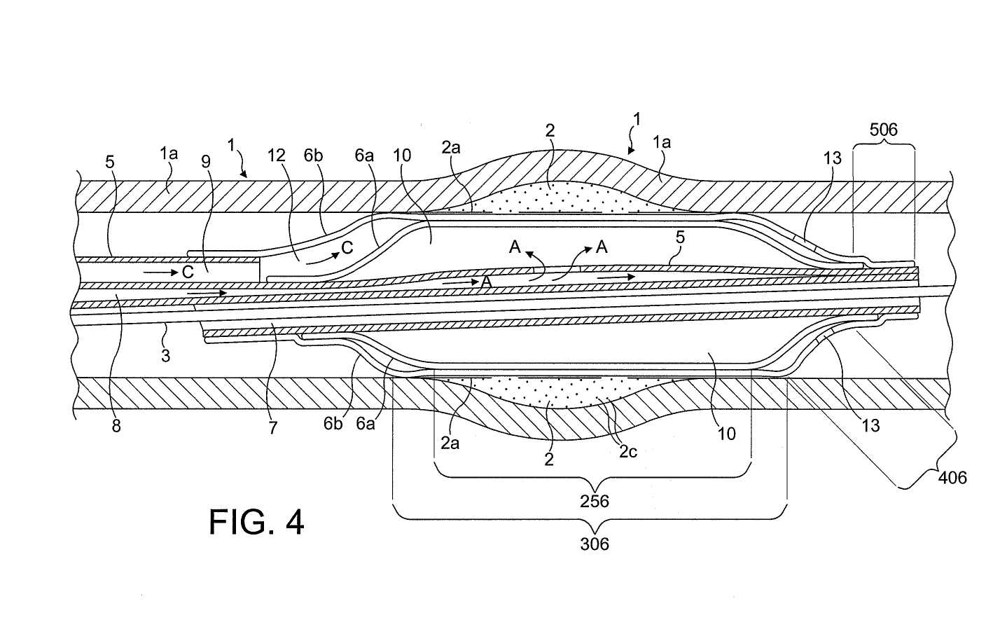

FIG. 4 is a side sectional view of the portion of the blood vessel and balloon angioplasty device shown in FIG. 2 following inflation of the inflatable element by supplying balloon inflation medium into a space enclosed by the dilatation balloon via a balloon inflation lumen extending through the catheter shaft of the balloon angioplasty device, to dilate the stenotic area of the vessel, an antegrade flushing fluid flow path being filled with flushing fluid but a distal portion of the antegrade flushing fluid flow path being closed by the distended dilatation balloon pressed against the membrane;

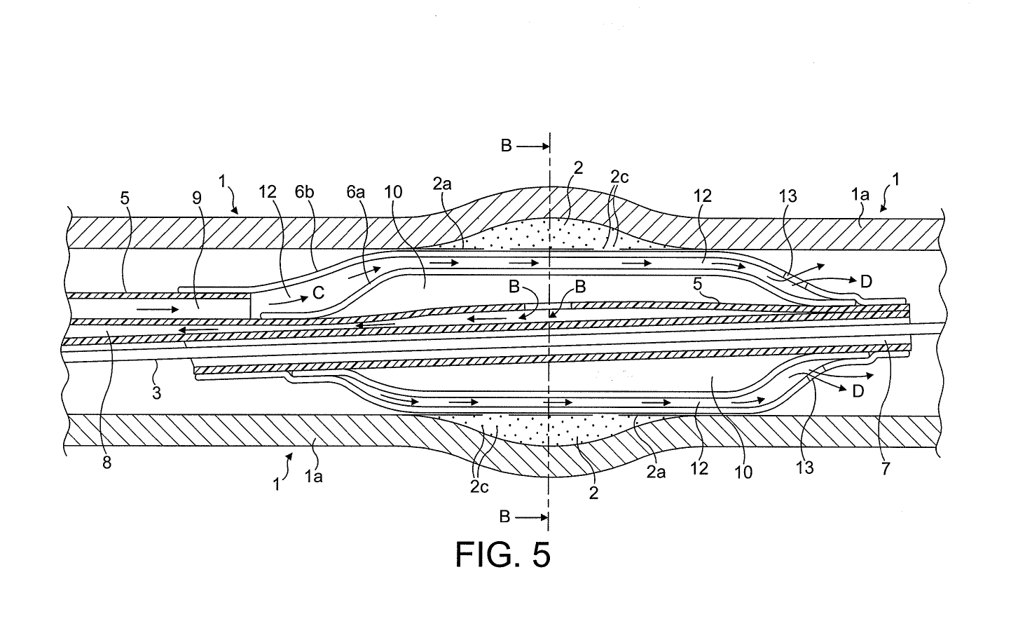

FIG. 5 is a side sectional view of a portion of the blood vessel and balloon angioplasty device shown in FIG. 4 following initial reduction of pressure in the dilatation balloon and pumping of pressurised flushing fluid through the antegrade flushing fluid flow path and its now opened distal portion located between the partially deflated dilatation balloon and the membrane so that the flushing fluid flows into the vessel distal to the treated stenotic lesion through at least one opening located at or near the distal end of the membrane, the membrane continues to be pressed by the pressure of the flushing fluid against at least the treated stenotic area of the vessel;

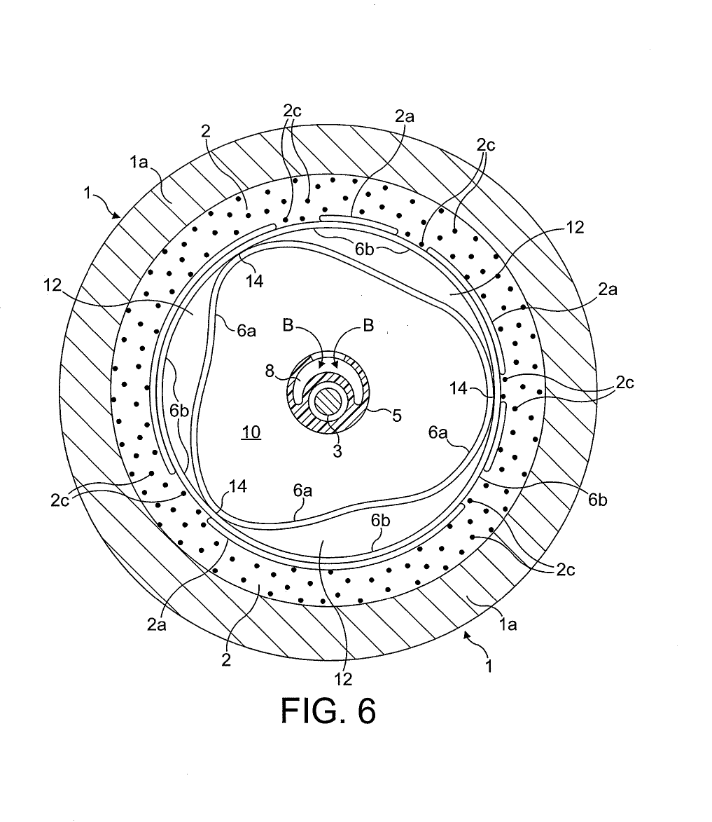

FIG. 6 is an enlarged cross-sectional view taken along the line B-B of FIG. 5 to more clearly illustrate how the membrane is maintained in contact with the treated stenotic lesion to prevent escape of debris from the treated stenotic lesion;

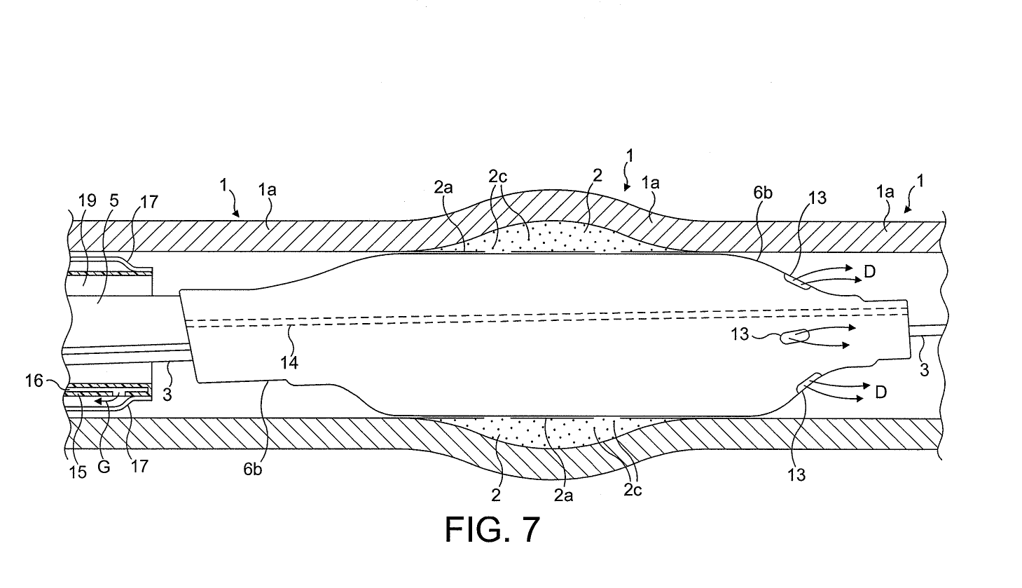

FIG. 7 is the same view as shown in FIG. 5 but with a longitudinal cross-section taken only through a portion of the blood vessel and distal end section of the drainage sheath thereby better illustrating both the antegrade flow of flushing fluid into the vessel from the openings at or near the distal end of the membrane and the contact of the membrane with the treated stenotic lesion;

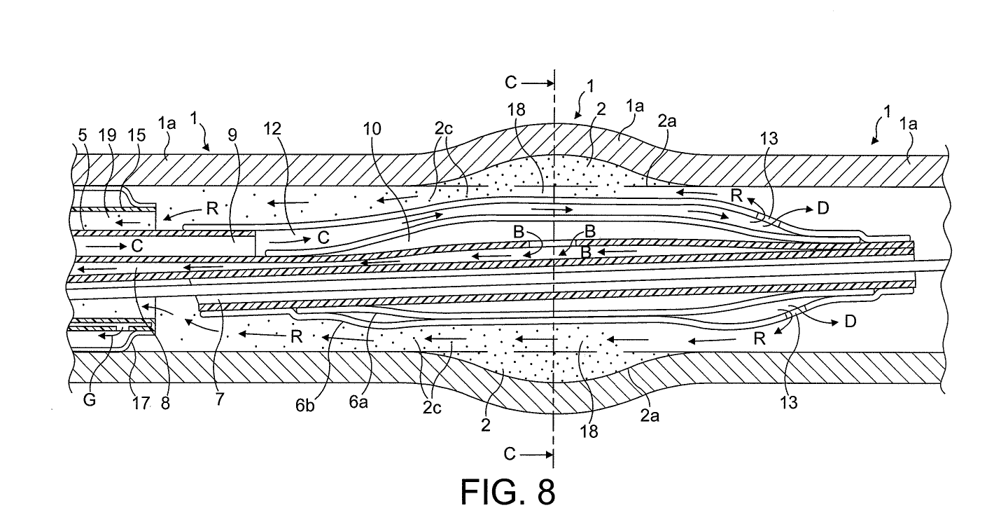

FIG. 8 is a side sectional view of the portion of the blood vessel after dilation of the stenotic area of the vessel has been completed and flushing fluid which has entered the vessel through at least one opening in the membrane is flowing in a retrograde direction along a retrograde fluid flow path between the membrane and the vessel wall after the membrane has been at least partially pulled away from the vessel wall by the collapsing or collapsed dilatation balloon. FIG. 8 also shows flow of retrograde flowing flushing fluid and entrained debris into the lumen of the drainage sheath, an inflatable cuff at the distal end of the drainage sheath having been inflated to restrict or block the antegrade flow of blood and the retrograde flow of flushing fluid between the vascular wall and the inflated cuff of the drainage sheath;

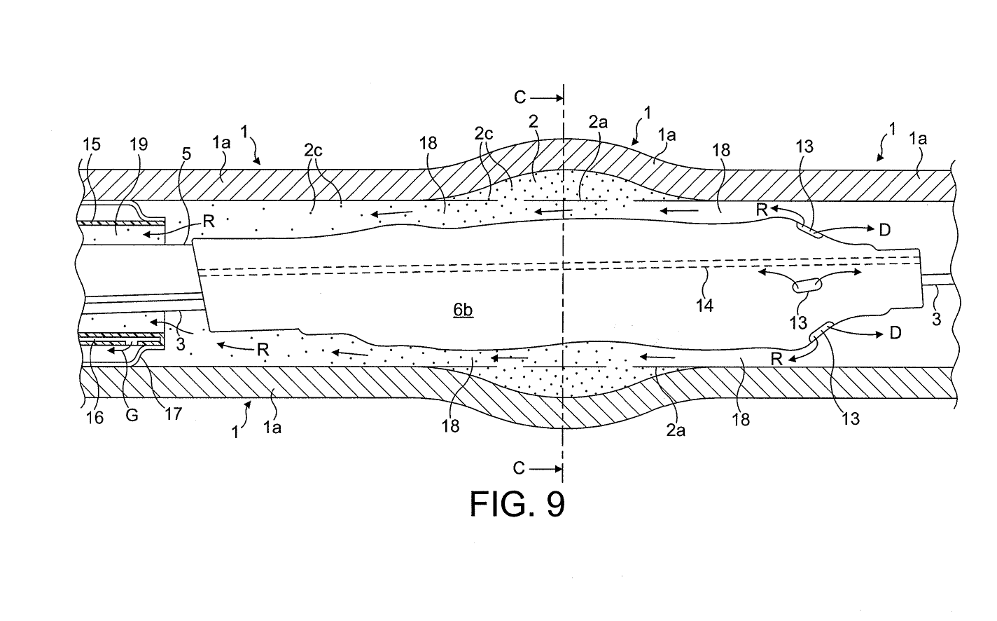

FIG. 9 is the same view as FIG. 8, but showing a longitudinal cross-section taken only through the portion of the blood vessel and distal end section of the drainage sheath, thereby better illustrating the retrograde flow of flushing fluid;

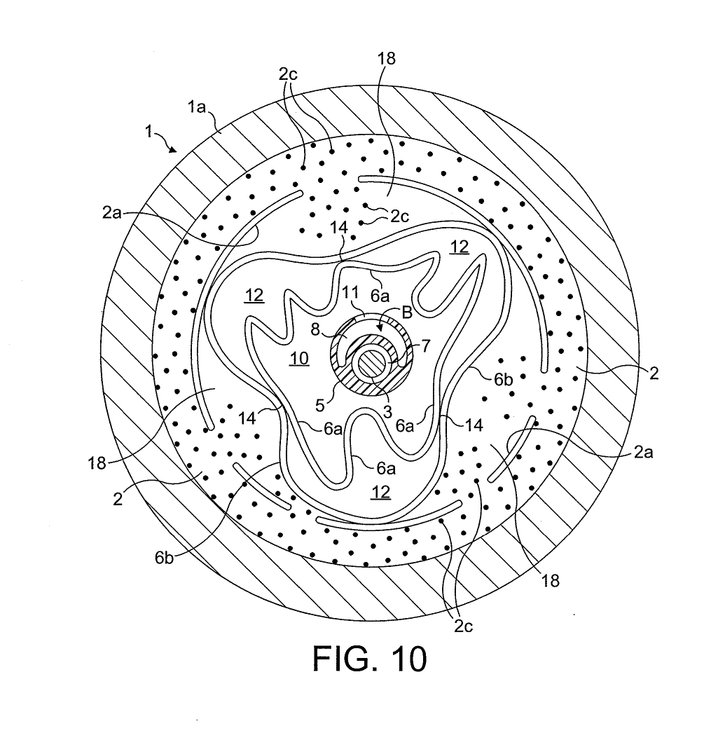

FIG. 10 is a cross sectional view of the device and blood vessel illustrated in FIGS. 8 and 9 taken along the line C-C to clearly illustrate the antegrade flow paths for the flushing fluid between the dilatation balloon and the membrane and the retrograde flow paths between the membrane and the treated stenotic lesion which allow for the retrograde flow of at least a portion of the flushing fluid that has entered the vessel through the openings in the membrane;

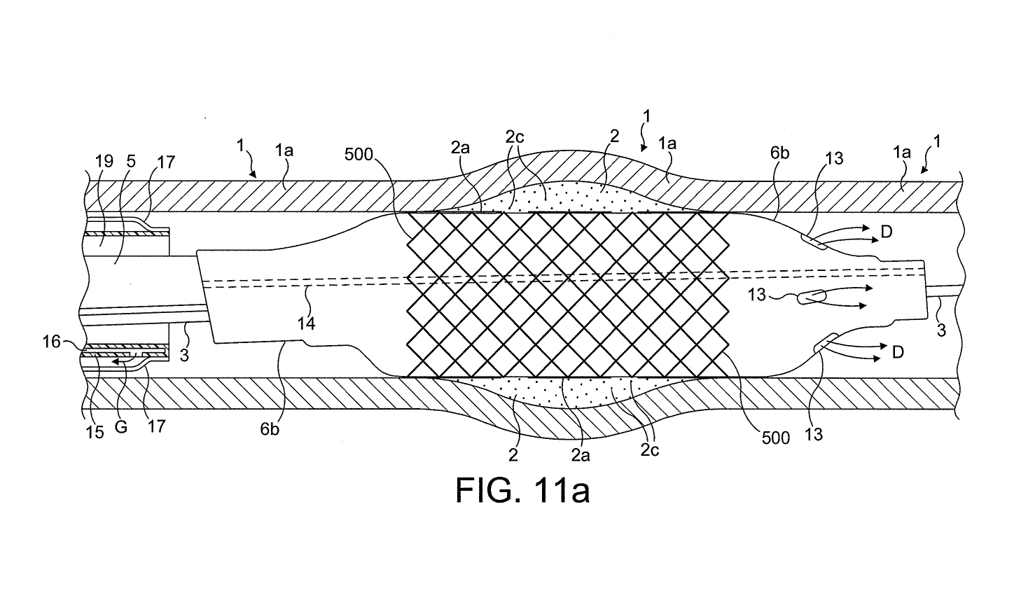

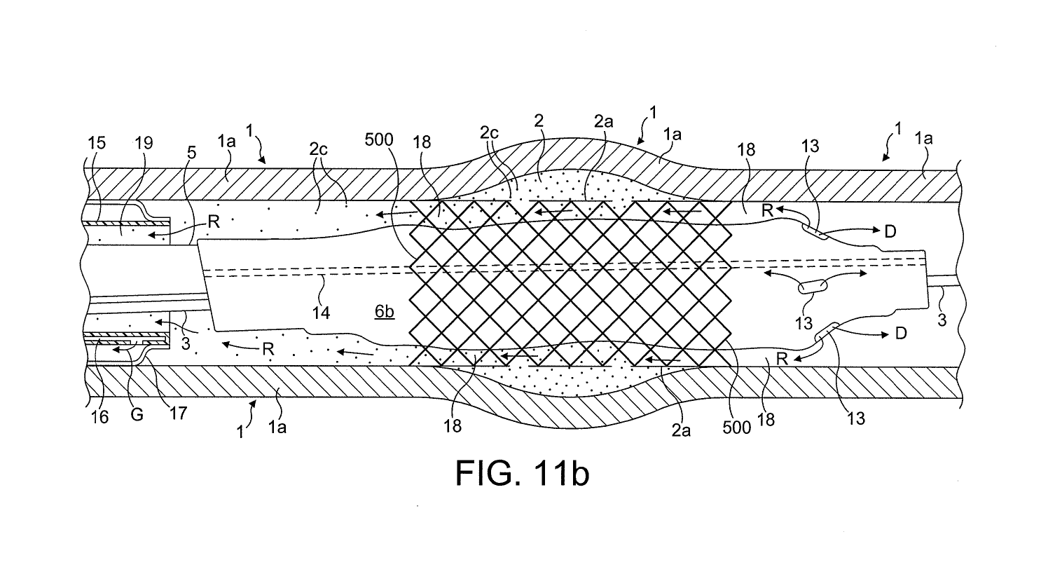

FIG. 11a is similar to FIG. 7, except that it illustrates that a stent pre-mounted on the inflatable element of the device has been delivered and deployed across the dialated stenotic region of the treated vessel by the balloon angioplasty device of the invention;

FIG. 11b is similar to FIG. 9, except that it illustrates the retrograde flow of flushing fluid and entrained debris along the retrograde flushing fluid flow path following stent deployment as shown in FIG. 11a;

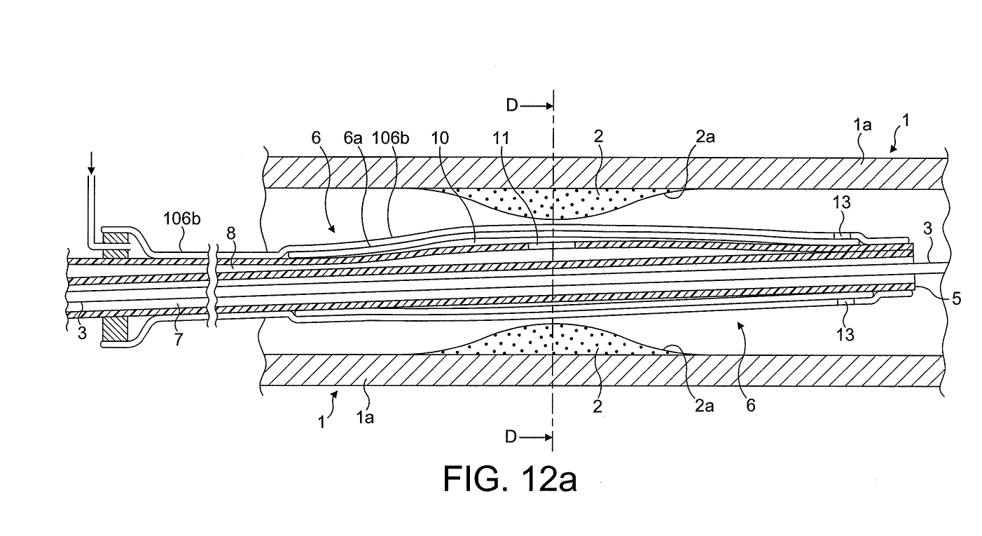

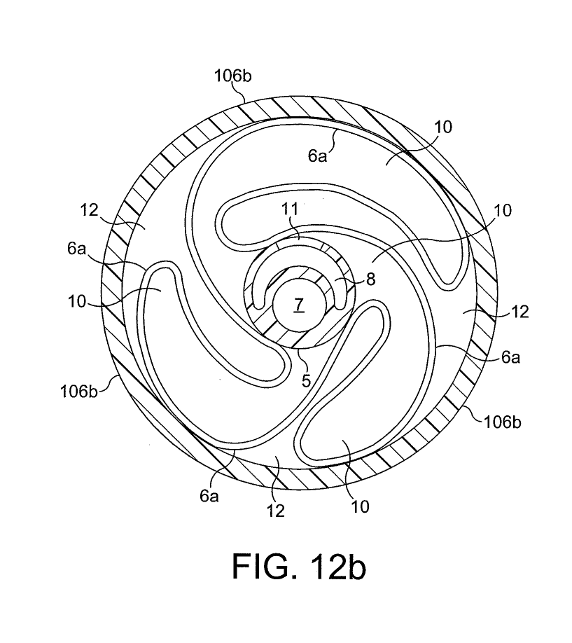

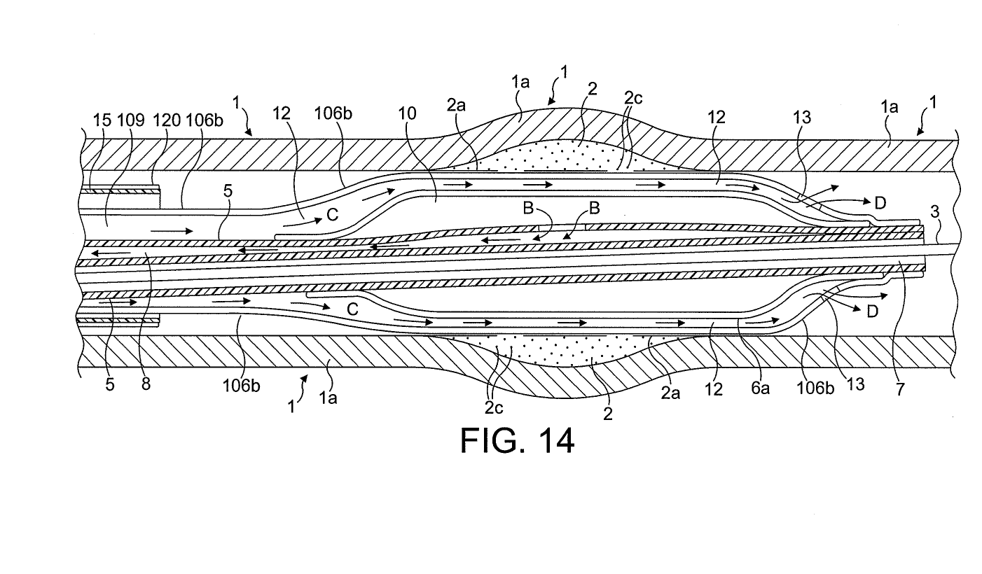

FIG. 12a illustrates an embodiment in which the guidewire lumen extends along the entire length of the catheter shaft and the shaft has a long stretchable membrane extending in a longitudinal direction around both the catheter shaft and the dilatation balloon thereby forming a proximal portion of the antegrade flushing fluid flow path between the catheter shaft and the stretchable membrane, the entire antegrade flushing fluid flow path is shown closed in this FIG. 10 and is shown opened by the pressure of the flushing fluid in FIG. 14;

FIG. 12b is an enlarged cross-sectional view taken along the line D-D of FIG. 12a with the blood vessel omitted for clarity;

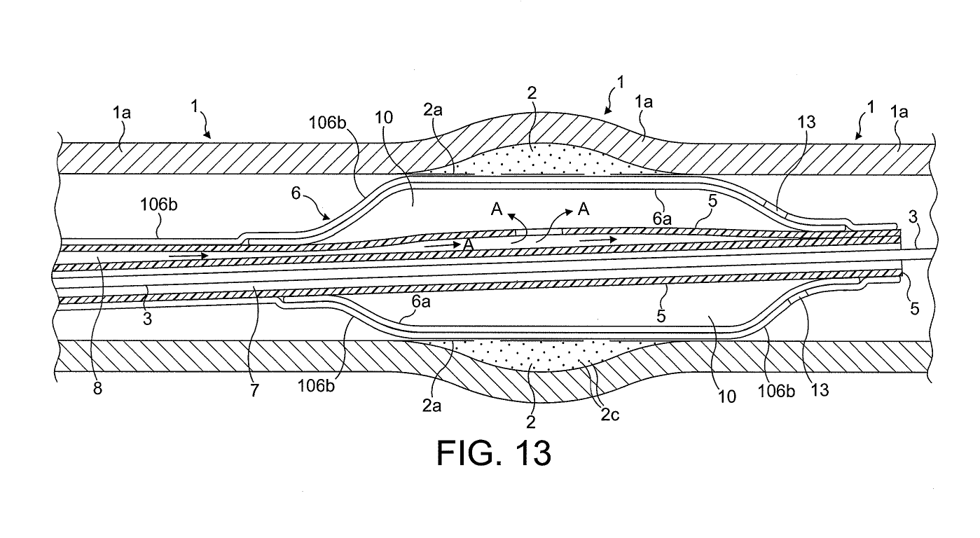

FIG. 13 is a side sectional view of the portion of the blood vessel and balloon angioplasty catheter shown in FIGS. 11 and 12 following inflation of the inflatable element by supplying balloon inflation medium into a space enclosed by the dilatation balloon via a balloon inflation lumen extending through the shaft of the balloon angioplasty device, to dilate the stenotic area of the vessel;

FIG. 14 is similar to FIG. 13 except that pressure of the fluid in the dilation balloon has been reduced and the long stretchable membrane around the catheter shaft has been distended by the pressure of flushing fluid which is pumped between the stretchable membrane and the catheter shaft, thereby opening the proximal portion of the antegrade flushing fluid flow path and enabling flow of pressurised flushing fluid between the stretchable membrane and the dilatation balloon, the pressure of the antegrade flowing flushing fluid on the stretchable membrane being sufficient to continue to press the stretchable membrane against the treated stenotic lesion, the FIG. 14 also showing the distal end portion of a modified drainage sheath which includes a stretchable sleeve extending over its outer surface;

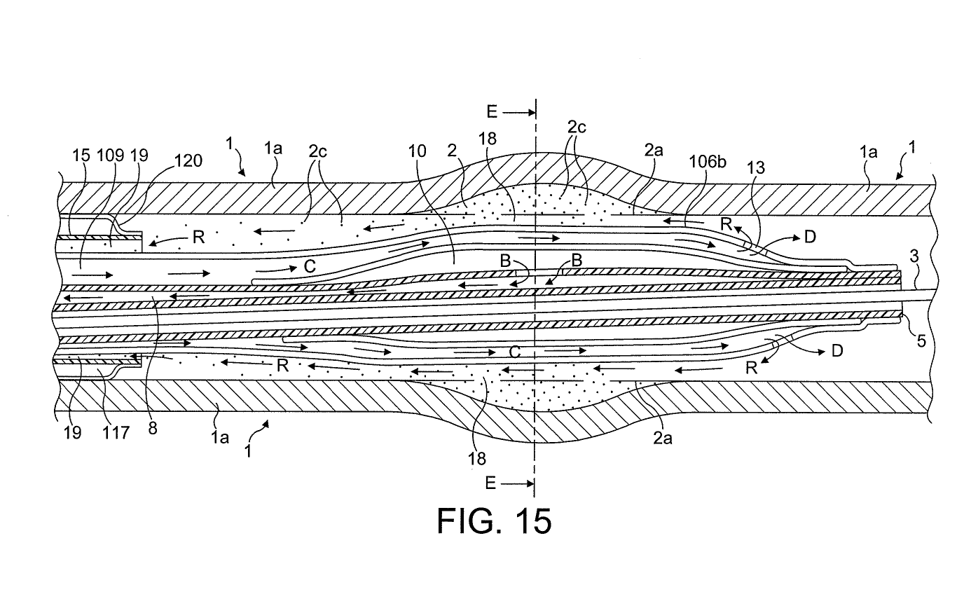

FIG. 15 is similar to FIG. 14, except that dilation of the stenotic area of the vessel has been completed and the retrograde fluid flow path between the membrane and the treated stenotic lesion has been achieved by combining further reduction of pressure in the dilatation balloon with reduction of flushing fluid pressure to a level at which pressure of flushing fluid on the membrane is no longer sufficient to press the membrane against the treated stenotic lesion and the stretchable membrane has partially regained its unstretched state to an extent at which the membrane is spaced away from both the stenotic lesion and the dilatation balloon thereby preserving both the antegrade fluid flow path for pumping flushing fluid between the balloon and the membrane and forming a retrograde fluid flow path between the membrane and the treated stenotic lesion, FIG. 15 also showing flushing fluid and entrained debris flowing in the lumen of the modified drainage sheath after the stretchable sleeve of the drainage sheath has been inflated to restrict or block the antegrade flow of blood and the retrograde flow of flushing fluid and debris around the drainage sheath;

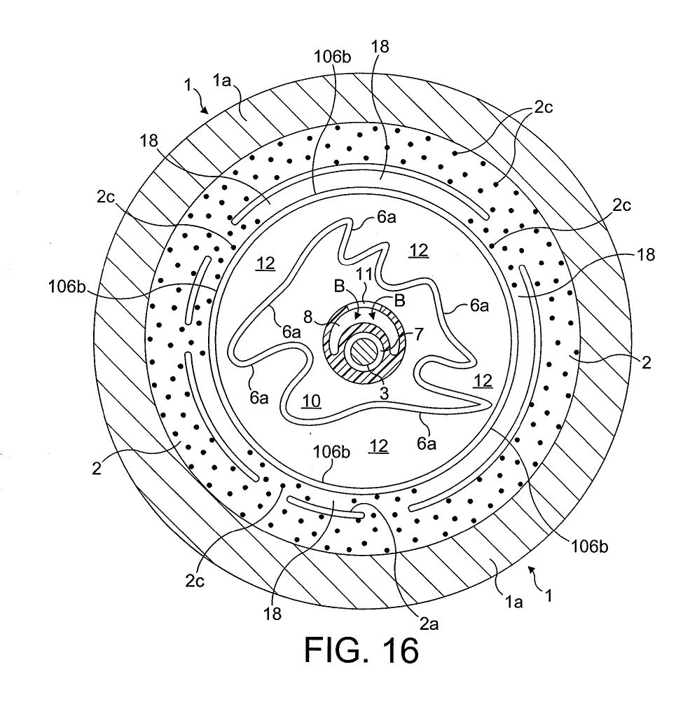

FIG. 16 is an enlarged cross-sectional view taken along the line E-E of FIG. 15, and

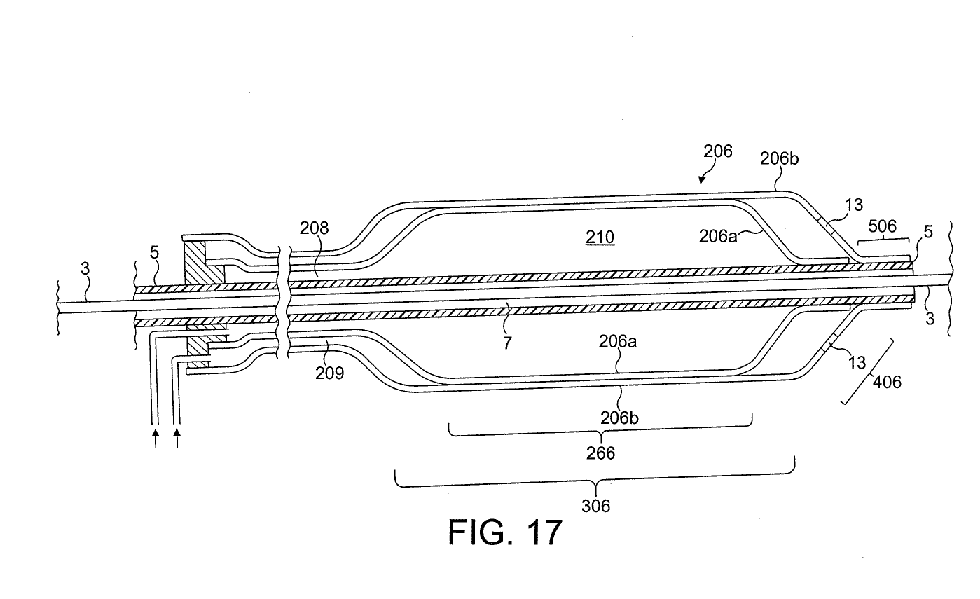

FIG. 17 is a side sectional view of another modified embodiment of balloon angioplasty device which is similar to the embodiment of FIG. 12a, except that the balloon inflation lumen within the catheter shaft has been replaced by a balloon inflation lumen which is formed integrally with the dilatation balloon and extends proximally from the balloon around the catheter shaft. A membrane common to the balloon and its inflation lumen is attached proximally to a proximal end portion of the catheter shaft so that now both balloon inflation and flushing fluid supply lumens are formed by the membranes extending around the catheter shaft, which now comprises only a guidewire lumen, the guidewire lumen extending within and along the entire length or substantially the entire length of the catheter shaft.

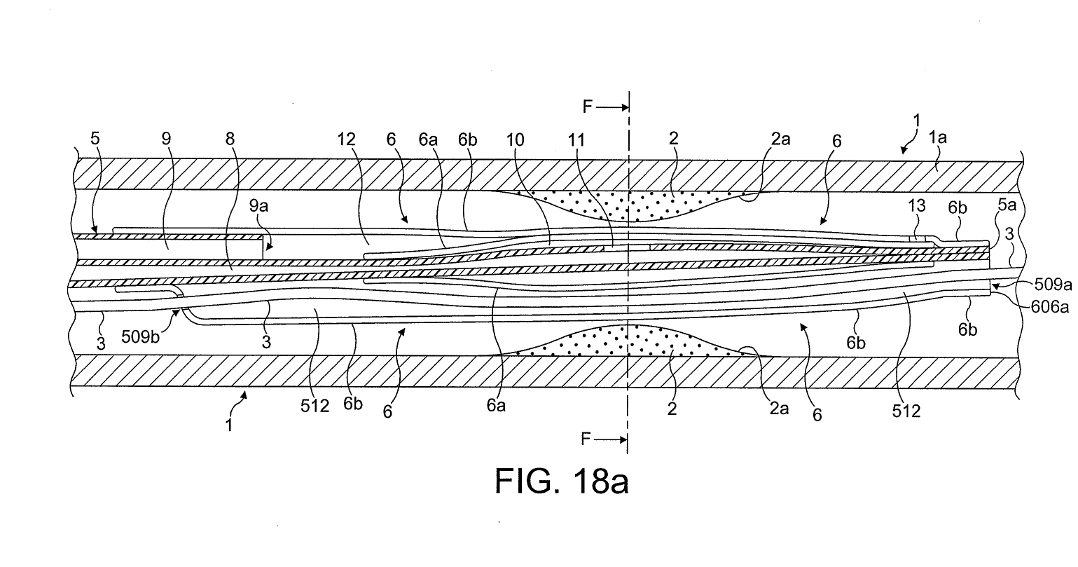

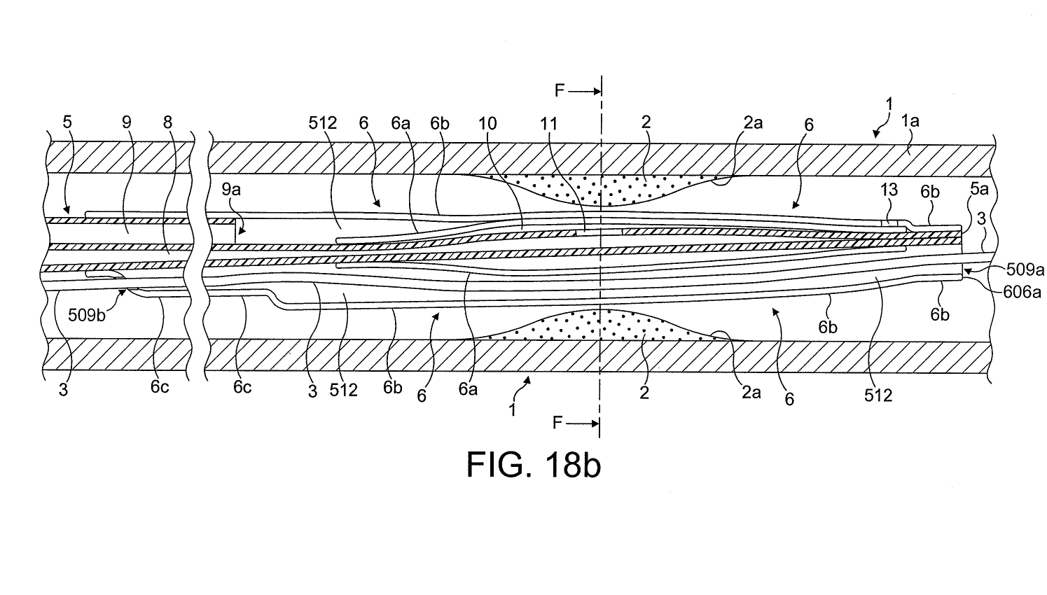

FIG. 18a is similar to FIG. 2 except that the catheter shaft does not include a guidewire lumen, the guidewire extending between the wall of the dilatation balloon and the membrane of the inflatable element;

FIG. 18b is substantially similar to FIG. 18a, except that it illustrates that a distal end portion of the membrane of the inflatable element, which extends around the dilatation balloon has a larger diameter than a substantially longer smaller diameter portion of the membrane which extends proximally around the catheter shaft for a length required to introduce the balloon angioplasty device of the invention into a coronary or peripheral artery without using a dedicated guidewire lumen extending proximally through a certain length of the catheter shaft;

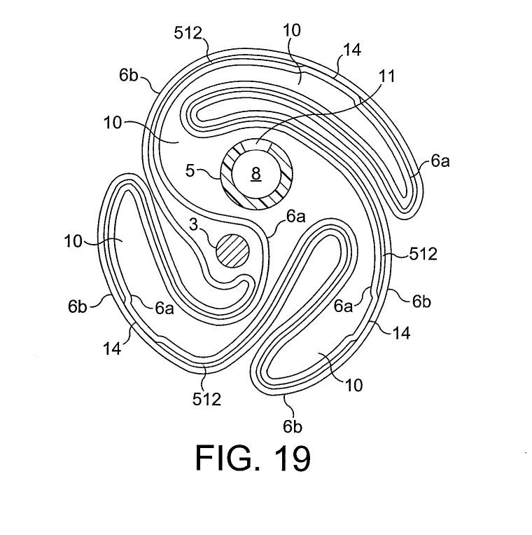

FIG. 19 is an enlarged cross-sectional view taken along the lines F-F shown in FIGS. 18a and 18b and illustrating the location of the guidewire within the furled inflatable element of the balloon angioplasty device;

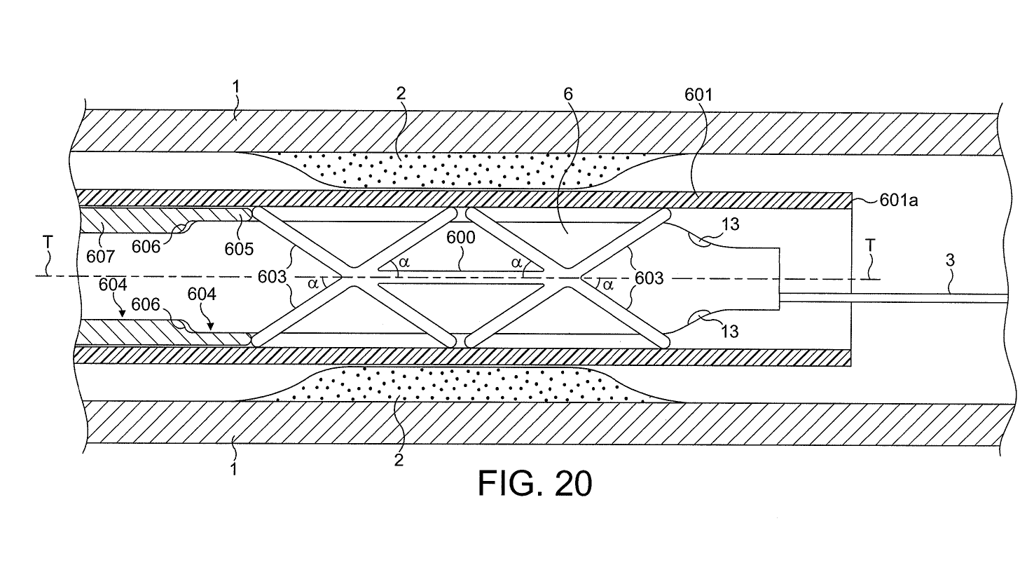

FIG. 20 is a side sectional view of a portion of a blood vessel having a stenotic lesion which has been crossed by a self-expanding stent which is disposed around the balloon angioplasty device of the invention, with the stent being restrained from expanding by a tubular advancing catheter;

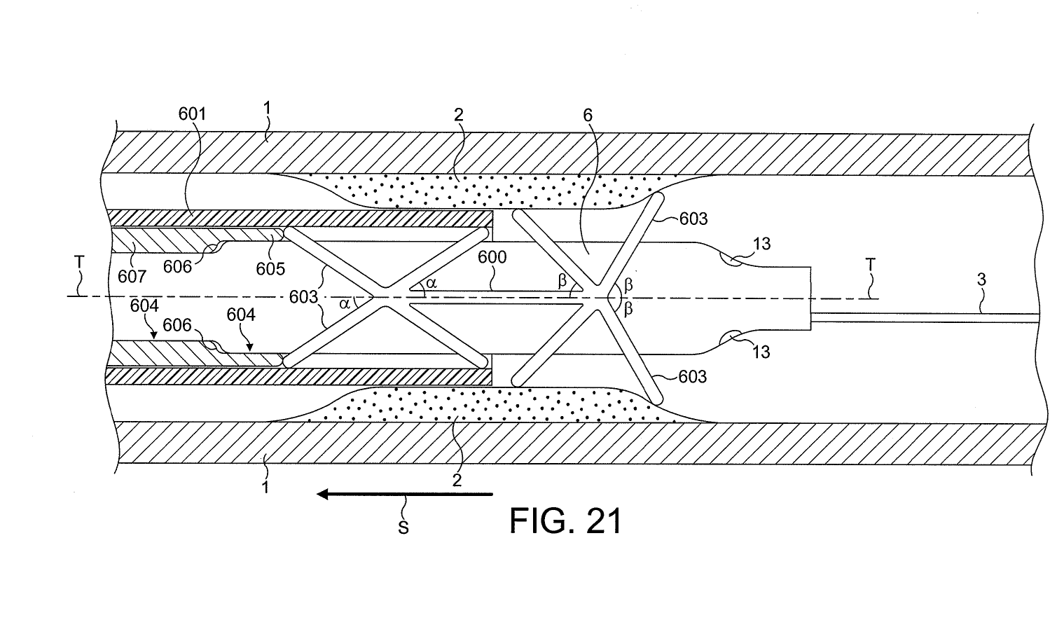

FIG. 21 shows the side sectional view of FIG. 20 with the tubular advancing catheter being partially withdrawn with respect to the stenotic lesion and the self-expanding stent, thereby allowing struts of the self-expanding stent to expand and contact the stenotic lesion;

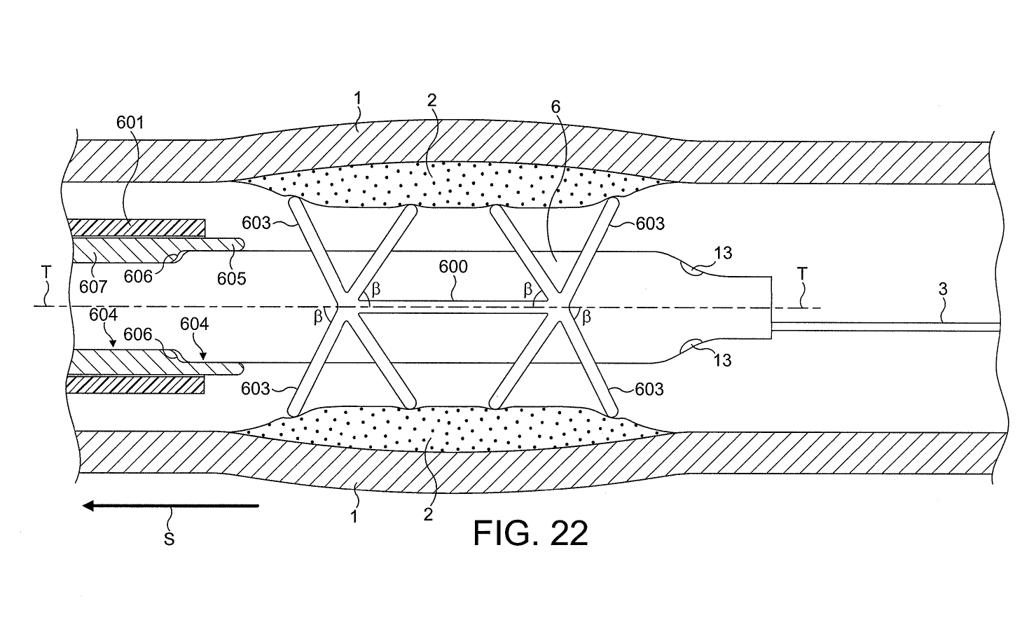

FIG. 22 shows the side sectional view of FIGS. 20 and 21 after the self-expanding stent has been fully released from the restrains of the tubular advancing catheter and the struts of the stent are in contact with the stenotic lesion and,

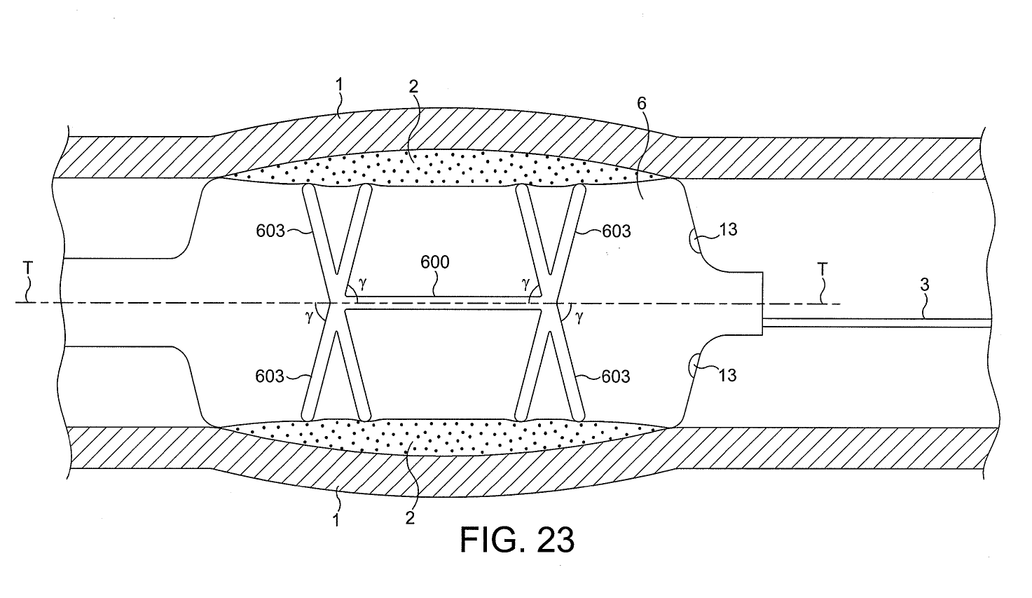

FIG. 23 shows the side sectional view of FIGS. 20 to 22 during inflation of the angioplasty balloon so as to dilate the stenotic lesion and allow the struts of the self-expanding stent to assume their final, fully expanded, position.

Reference is made in this specification to “distal” and “proximal” ends and to flow of fluid in an “antegrade” and “retrograde” direction. For the purpose of this specification, the distal end is considered to refer to the end of the device which is advanced into the vessel in the body of a patient and, the proximal end is the opposite end of the device which remains outside the body of the patient and is connected to fluid pumping and suction devices. Antegrade flow refers to a direction of fluid flow from the proximal end towards the distal end. Similarly, retrograde flow refers to a direction of flow in the opposite direction, i.e. from the distal to the proximal end of the device. It will also be appreciated that the terms “device” and “catheter” are used interchangeably throughout the specification.

FIG. 1 illustrates a side sectional view of a portion of a blood vessel 1 which may be a coronary or peripheral artery, a vascular bypass graft or arteriovenous shunt. The blood vessel 1 has been partially occluded by the presence of an atherosclerotic plaque 2 in the wall 1a of the vessel 1. The plaque 2 restricts the flow of blood through the vessel 1. Atherosclerotic plaques usually develop over a long period of time within the wall 1a of an artery and are frequently referred to as “stenotic lesions” as soon as they start to compromise blood flow through the artery. Usually atherosclerotic plaque has a fibrotic cap 2a and rupture or erosion of such fibrotic cap is frequently associated with acute thrombosis of the artery.

The device, according to any of the embodiments of the invention, can be used to dilate the vessel 1 in the region of the stenotic lesion 2 so as to open up or widen the vessel thereby restoring proper flow of blood through the vessel 1 and across the region of the vessel occupied by the stenotic lesion 2. Typically, the location and degree of stenosis (narrowing) in, for example, the coronary artery is ascertained using conventional angiography or angiographic techniques. If the degree of stenosis is found to be severe enough to warrant a balloon angioplasty procedure, then a conventional guidewire 3 is advanced across the stenotic region 2, as shown in FIG. 1.

Once the guidewire 3 has been located in the desired position, the distal end of a hollow tubular catheter shaft 5 of the balloon angioplasty device is advanced over the proximal end of the guidewire 3 and the device is tracked along the guidewire into and along the vessel 1 until an inflatable element 6 is situated across the stenotic lesion 2, as shown in FIG. 2. FIG. 2 shows an embodiment in which the inflatable element 6 is located or mounted on the catheter shaft 5 a short distance proximal to the distal end of the shaft 5. Although it is possible for the catheter shaft 5 to have a guidewire lumen extending all the way through it from the distal end to the proximal end thereof, the shaft 5 may well be formed with a guidewire lumen 7 which extends longitudinally over only a limited distal length of the shaft 5 to facilitate the use of a shorter guidewire and to enable rapid placement or exchange of the angioplasty catheter. As illustrated in FIG. 2, the catheter shaft 5 is provided with a relatively short guidewire lumen 7 that extends proximally from the distal end of the shaft 5. As will be appreciated by those skilled in the art, the proximal end portion of the guidewire 3 always extends proximally from the proximal end of the guide catheter (not shown) for a length necessary to accomplish fast advancement and exchange of angioplasty devices.

As will be explained in more detail later, the inflatable element 6 of the device comprises a substantially fluid impermeable elongate dilatation balloon 6a and a substantially fluid impermeable membrane 6b extending in a longitudinal direction around the balloon 6a. In addition to the guidewire lumen 7, the catheter shaft 5 includes a balloon inflation lumen 8 extending through it from the proximal end or end portion of the shaft 5, to enable the balloon 6a to be inflated and, a flushing fluid supply lumen 9 through which flushing fluid can be pumped into a potential or normally closed fluid flow path between the balloon 6a and the membrane 6b of the inflatable element 6 and through this fluid path into the vessel 1 through flushing fluid openings 13 located at or near the distal end of the membrane 6b. It will be appreciated that, when balloon angioplasty device of the invention is properly placed across the stenotic lesion to be treated, flushing fluid openings 13 in the membrane 6b are located distally to the stenotic lesion 2 (see FIGS. 2, 4, 5 and 7).

FIG. 2 shows a longitudinal cross-sectional view of a most distal end portion of the balloon angioplasty catheter located in the vessel 1 of a patient with the inflatable element 6 in a deflated condition and extending in a longitudinal direction around the most distal end portion of the catheter shaft 5. As already mentioned above, the inflatable element 6 comprises the dilatation balloon 6a and the membrane 6b. The balloon 6a extends in a longitudinal direction around the catheter shaft 5 and is mounted to it. In the embodiment shown in FIG. 2, each end of the dilatation balloon 6a is sealingly attached to the distal end portion of the catheter shaft 5, the proximal end of the dilatation balloon 6a being attached to the catheter shaft 5 at a point close to or distal to the distal end of the flushing fluid supply lumen 9. An inner space 10 enclosed by the dilatation balloon 6a is in communication with the balloon inflation lumen 8 via an opening 11 in the wall of the catheter shaft 5 located in-between the ends of the dilatation balloon 6a so that inflation medium which may be a liquid contrast medium, saline solution or a gas such as helium, may be supplied through the inflation lumen 8 and via the opening 11 into the said space 10 bounded by the balloon 6a to inflate the inflatable element 6.

The membrane 6b of the inflatable element 6 extends in a longitudinal direction around the dilatation balloon 6a and both ends of the membrane are attached to the catheter shaft 5. In the embodiment shown in FIG. 2, the proximal end of the membrane 6b extends proximally beyond the proximal end of the dilatation balloon 6a and is attached to the shaft 5 at a location proximal to the distal end of the flushing fluid supply lumen 9 so that the flushing fluid supply lumen 9 is in communication with at least one potential fluid flow path 12 extending between the balloon 6a and the membrane 6b.

As previously mentioned, the membrane 6b has at least one flushing fluid opening 13 at, or close to, its distal end (see FIGS. 2,4 and 5) so that fluid pumped through the flushing fluid supply lumen 9 into the potential fluid flow paths 12 between the membrane 6b and the dilatation balloon 6a may flow through the or each flushing fluid opening 13 in the membrane 6b into the vessel 1 distal to the stenotic lesion 2, as will become apparent from the following description relating to the operation of the device.

In the embodiments shown in FIGS. 2 through 10, the flushing fluid supply lumen 9 and the potential fluid flow path(s) 12 between the dilatation balloon 6a and the membrane 6b forming an antegrade flushing fluid flow path, the flushing fluid supply lumen 9 forming a proximal portion of such antegrade flushing fluid flow path and the potential flow path(s) between the dilatation balloon 6a and the membrane 6b forming the distal portion of the antegrade flushing fluid flow path.

The distal portion of the antegrade flushing fluid flow path located between the dilatation balloon 6a and the membrane 6b is shown closed in FIGS. 2 through 4 and is shown open in FIGS. 5 through 10, as will be described in more detail later.

Reference will now be made to FIG. 3, which shows an enlarged cross-section taken along the line A-A in FIG. 2 of the device (with the vessel 1 and guidewire 3 omitted for clarity). It will be appreciated from this drawing, that the balloon 6a and the membrane 6b of the inflatable element 6 are furled up around the outside of the catheter shaft 5 to facilitate the advancement of the shaft 5, together with the inflatable element mounted thereon, into the vessel 1 and across the stenotic lesion 2 (not shown in FIG. 3). When the inflatable element 6 is furled, the wall of the balloon 6a and the membrane 6b generally lie in contact with each other so that the fluid flow paths 12 between the balloon 6a and the membrane 6b are collapsed or not open in this furled state of the device thereby forming only potential fluid flow paths that are normally closed but become open during use of the device in the process of treatment of the stenotic lesion. The normally closed fluid flow paths 12 open in response to the pumping of flushing fluid along the flushing fluid supply lumen 9 which flows via said flow paths 12 and into the vessel 1 through openings 13 located at or near the distal end of the membrane 6b, as will be described in more detail later.

As can be seen in FIG. 3, the dilatation balloon 6a and the membrane 6b of the inflatable element 6 are attached to each other at three separate locations 14 spaced circumferentially around the axis of the catheter shaft and which extend longitudinally along the length of the inflatable element 6 for reasons which will become apparent. Each location where the balloon 6a and membrane 6b are joined to each other may be in the form of longitudinally extending elongate strips (one such strip 14 being illustrated in dashed lines in FIGS. 7 and 9) which may be continuous, i.e. they extend longitudinally along the length of the inflatable element as shown or, they may be discontinuous in which case the strips may have breaks in them where the balloon 6a and membrane 6b are not attached to each other. For example, each longitudinally extending connection 14 could be formed by a series of spaced dots or spot welds extending in a longitudinal direction where the balloon 6a and the membrane 6b are connected or welded to each other at spaced locations. Although reference is made to “strips”, it will be appreciated that the width of the attachment between the membrane and the dilatation balloon in a circumferential direction may be minimal, the balloon and membrane being attached along a join-line having no appreciable width in the circumferential direction.

Although in this embodiment, the balloon 6a and membrane 6b are attached to each other at just three radially spaced locations around the circumference of the shaft, it will be appreciated that there could be more or fewer points 14 of attachment spaced from each other around the circumference of the catheter shaft 5.

The balloon 6a and the membrane 6b may both be made of a substantially non-stretchable material and are shown to be formed from such a material in the majority of the drawings, especially in FIG. 3, which shows the inflatable element 6 in a furled state. However, it is also envisaged that the membrane 6b could also be made from a material which stretches during inflation so that only the dilatation balloon 6a is in a furled state inside a stretchable membrane when the angioplasty device is ready to be advanced into a vessel to be treated. The stretchable membrane 6b may be formed from silicon resin or other suitable material. This embodiment is described in more detail later with reference to FIGS. 11 to 16 of the accompanying drawings.

In one embodiment of the invention, the membrane 6b of the inflatable element, when inflated by the dilatation balloon 6a, has a maximum diameter section 306 which extends in a longitudinal direction around a maximum diameter section 266 of the dilatation balloon 6a, the maximum diameter section of the membrane continues distally into a transitional segment 406 of the membrane 6b which becomes progressively smaller in diameter as it converges towards the catheter shaft and finally continues into a minimum diameter segment 506 of the membrane which is connected to the catheter shaft 5, the transitional segment 406 of the membrane 6b including the or each flushing fluid opening(s) 13 of the membrane as shown best in FIGS. 4 and 5.

The location of the or each flushing fluid opening(s) 13 in the membrane 6b along its transitional segment 405 is advantageous in preventing premature rupture of the membrane since any pressure applied to the membrane 6b is applied to a smaller diameter surface along the transitional segment 405 of the membrane thereby creating smaller tearing forces along the transitional segment 405 as compared to tearing forces which may become applied, or transmitted, to the membrane by the same fluid pressure applied to the maximum diameter section 306 of the membrane.

In the preferred embodiment of the invention best shown in FIG. 4, the maximum diameter section 306 of the membrane 6b extends distally along the catheter shaft 5 at least a short distance longer than the diameter section 266 of the dilatation balloon 6a so that the membrane and the dilatation balloon are together able to withstand without rupture a certain maximum predetermined pressure of inflation medium within the dilatation balloon.

In the most preferred embodiment of the invention, shown best in FIGS. 4 and 17, the maximum diameter section 306 of the membrane both distally and proximally extends beyond corresponding distal and proximal ends of the maximum diameter section 266 of the dilatation balloon, thereby further increasing the ability of the dilatation balloon and the membrane to withstand together a certain maximum predetermined pressure of the inflation medium within the dilatation balloon and at least the dilatation balloon being designed to rupture when the pressure within the balloon exceeds a certain predetermined burst pressure.

The catheter shaft 5 is received within a drainage sheath 15, which terminates distally proximal to the inflatable element 6 mounted on the catheter shaft 5 (see FIGS. 7 and 9). In the most preferred embodiment of the invention, the drainage sheath 15 is longitudinally movable along the catheter shaft 5 and is advanced into its proper position within vessel 1 either together with, or following advancement and proper placement of the catheter shaft 5. To enable or assist the flow of the flushing fluid in a retrograde direction across the treated stenotic area of the vessel and from the treated vessel into the drainage sheath 15, the proximal end portion of the lumen 19 of the drainage sheath 15 may be in communication with a suction device (not shown). In one embodiment, the drainage sheath 15 includes an inflation lumen 16 in communication with an inflatable member 17 such as an inflatable cuff or balloon mounted close to the distal end of the drainage sheath 15. The inflatable member 17 is inflated after the drainage sheath 15 has been positioned within the vessel 1 to prevent antegrade flow of blood or retrograde flow of flushing fluid and debris 2c between the inflatable element 17 of the sheath 15 and the vessel wall 1a, as will be explained.

Operation of the angioplasty device to dilate the vessel 1 and the stenotic lesion 2 located within the vessel 1 will now be described mainly with reference to FIGS. 4 to 10 of the accompanying drawings.

Once the angioplasty device is located in the vessel, as shown in FIG. 2, with the inflatable element 6 positioned across the region of the vessel 1 which has been occluded by the stenotic lesion 2, the dilatation balloon 6a is inflated by pumping inflation medium, such as X-ray contrast solution or other fluid, along the balloon inflation lumen 8 of the catheter shaft 5 and into the dilatation balloon 6a via the opening 11 in the catheter shaft 5 (as indicated by arrows “A” in FIG. 4). As the space 10 within the dilatation balloon 6a becomes distended with X-ray contrast solution, the pressure of such solution or other inflation medium pressure in the distended balloon 6a is applied or transmitted through the membrane 6b to the stenosis 2 and the vessel wall 1a to cause dilation of the vessel 1 in the region of the stenosis 2.

During the dilation procedure, the fibrotic cap 2a of the stenotic lesion may break causing debris 2c contained within the stenotic area to enter the vessel 1. This debris 2c may migrate distally along the vessel when the balloon 6a is deflated and if at the same time the membrane 6b is no longer pressed against the vessel wall 1a.

Debris 2c is prevented from escaping from the treated stenotic area and is then subsequently removed from the treated vessel 1, and even from the patient, by the procedure that will now be explained.

As the pressure of the fluid in the dilatation balloon 6a is reduced by initially withdrawing only a relatively small amount of fluid from the space 10 (as indicated by arrows “B” in FIGS. 5 and 6), flushing fluid is pumped through an antegrade flushing fluid flow path (in the direction of arrow “C” in FIGS. 5 and 8) and into the vessel 1, having flowed along the flushing fluid supply lumen 9 and between the balloon 6a and membrane 6b. The pressure of the flushing fluid in a distal portion 12 of the antegrade flushing fluid flow path located between the balloon 6a and membrane 6b and the pressure of the inflation fluid within the balloon 6a are carefully controlled so that the pressure of the flushing fluid is sufficient to both open the distal portion 12 of the antegrade fluid flow path located between the membrane 6b and the balloon 6a and keep the membrane 6b pressed against the vessel 1 and the stenotic lesion 2 thereby effectively sealing the treated stenotic lesion and preventing escape of debris into the vessel 1. The pressurised flushing fluid flows into the vessel 1 via the at least one flushing fluid opening 13 at or near the distal end of the membrane 6b of the inflatable element 6, as illustrated by arrow “D” in FIGS. 5,7,8 and 9.

The pressure of the flushing fluid between the membrane 6b and the balloon 6a assists deflation of the balloon 6a. Therefore, actively withdrawing fluid from the dilatation balloon 6a at this stage of the procedure may not be necessary and simply allowing a relatively small amount of fluid to escape from the dilatation balloon 6a may be sufficient to achieve partial deflation of the dilatation balloon 6a. The pumping of flushing fluid through the fluid supply lumen 9 and into the vessel 1 between the balloon 6a and the membrane 6b through the flushing fluid opening(s) 13 in the membrane 6b is continued until the pressure in the vessel 1 distal to the balloon 6a and the stenotic lesion 2 increases to a pressure at which a fluid pressure gradient is generated within the vessel 1 between a high pressure region distal to the balloon 6a and, a low pressure region proximal to the balloon 6a.

Substantial withdrawal of fluid or other inflation medium from the balloon 6a (as indicated by arrows “B” in FIGS. 8 and 10) results in substantially complete deflation and/or collapse of the dilatation balloon 6a. Since the dilatation balloon 6a and the membrane 6b are connected longitudinally to each other at various spaced locations 14 around the catheter shaft 5, the membrane 6b of the inflatable element 6 is pulled away from the vessel wall 1a and the stenotic lesion 2 by the collapse of the balloon 6a in at least one region extending over the locations 14 at which the balloon 6a and membrane 6b are connected to each other. This results in the formation of at least one fluid flow path 18 (see FIGS. 8 and 9) between the membrane 6b and the vessel wall 1a.

The above described fluid pressure gradient between vessel areas located distally and proximally to the dilatation balloon 6a causes at least a portion of the flushing fluid which has flowed into the vessel 1 in an antegrade direction to be re-directed, due to the fluid pressure gradient, over the membrane 6b and over the catheter shaft 5, in a retrograde direction, as indicated by arrow “R” in FIGS. 8 and 9.

As the membrane 6b of the inflatable element 6 has been pulled away from the stenotic lesion 2 and the vessel wall 1a over the regions 14 where the dilatation balloon 6a and membrane 6b are connected to each other, fluid flow paths 18 are formed between the membrane 6b and the vessel wall 1a along which fluid flows in a retrograde direction.

It will be appreciated that the method by which the membrane 6b and the balloon 6a are connected to each other is largely irrelevant so long as the connections fulfil their desired function of pulling the membrane 6b of the inflatable element 6 away from the vessel wall 1a and its treated stenotic area 2 when the dilatation balloon 6a is deflated and/or collapsed to form the fluid flow paths 18 for the retrograde flow of fluid between the membrane 6b and the vessel 1.

It should be emphasised once again that in order to prevent migration of debris 2c along the vessel 1 in a distal direction, balloon 6a should be collapsed and the membrane 6b pulled away from the vessel wall 1 only when the pressure of the flushing fluid in the treated vessel 1 distal to the stenotic lesion 2 is sufficiently high to assure retrograde flow of at least a portion of the flushing fluid between the treated stenotic lesion and the membrane 6b along fluid flow paths 18.

It will be appreciated that as soon as such fluid flow paths 18 are formed, at least a portion of the flushing fluid that has entered the vessel 1 through the flushing fluid openings 13 in the membrane 6b will flow in a retrograde direction along said fluid flow paths 18 due to the fluid pressure gradient between a high pressure region distal to the point of entry of the flushing fluid into the vessel 1 and a low pressure region proximal to the point of entry of the flushing fluid into the vessel 1. Therefore, migration of debris in a distal direction is prevented.

As the flushing fluid flows along the fluid flow paths 18 over the membrane 6b, it entrains debris 2c released from the stenotic lesion 2. This debris 2c is carried by the fluid in a retrograde direction into a lumen 19 in the sheath 15 defined by an annular space between the catheter shaft 5 and the drainage sheath 15. The debris 2c is either retained within the annular space 19 within the drainage sheath 15 or, flows out of the patient through an opening at or near the proximal end of the sheath 15 together with the retrograde flow of flushing fluid.

Although the retrograde flow of fluid between the membrane 6b and the vessel wall 1a is preferably achieved by the generation of a pressure gradient in the vessel 1 as explained above, it may alternatively or additionally be assisted by the presence of a suction device (not shown) in communication with the lumen 19 in the sheath 15, which is operable to cause fluid and debris 2c in the vessel 1 to be drawn or sucked into the lumen 19 in the drainage sheath 15.

It will be appreciated that a stable flow of pressurised flushing fluid must be established and that all fluid flow pressures must be monitored throughout the procedure to ensure that when the retrograde flow of flushing fluid is taking place, preferably only saline solution, or other flushing fluid pumped into the vessel 1 through the openings 13 in the membrane 6b flows in a retrograde direction over the membrane 6b and entrains debris into the drainage sheath 15.

The drainage sheath 15 may be provided with an inflatable member 17 such as a cuff or balloon mounted adjacent to its distal end which, when inflated, prevents antegrade flow of blood and retrograde flow of fluid, and any debris 2c entrained therein, around the inflatable element of the sheath 15. Inflatable member 17, when carefully inflated to a relatively low pressure may also be used to locate the sheath 15 within the vessel 1. A balloon or cuff inflation lumen 16 extends within the wall of the sheath 15 and connects the inflatable member 17 to an inflation device (not shown) for supplying inflation medium into the inflatable member 17. The preferred inflation medium for inflating the inflatable member 17 is helium or other blood or saline soluble gas since helium or other suitable gas will not cause formation of an embolism if it leaks into the vessel 1 out of a ruptured inflatable element 17. Liquid X-ray contrast material may alternatively be used. The use of a gaseous inflation medium which is soluble in blood is preferable because a capillary effect is avoided and short inflation and deflation times are enabled.

The inflation lumen 16 of the drainage sheath 15 is connected to a pump (not shown) for supplying inflation medium into the inflatable member 17, as shown in FIGS. 7 and 9 by arrow “G”. In one embodiment, a syringe could be used as a pump for pumping the inflation medium into the inflatable member 17.

FIG. 12a illustrates the modified embodiment of the invention mentioned above and in which the guidewire lumen 7 extends along the entire length of the catheter shaft 5 and the shaft 5 has a long stretchable membrane 106b which extends in a longitudinal direction around the shaft 5 and the dilatation balloon 6a so that the entire antegrade flushing fluid flow path is formed integrally by the stretchable membrane 106b. This embodiment has the advantage that a separate flushing fluid supply lumen in the catheter shaft 5 is not required.

FIGS. 12a to 16 illustrate how a physician, using the modified embodiment of the device shown in FIG. 12a, would be able to use pressurised flushing fluid to stretch or distend the long stretchable membrane 106b around the catheter shaft 5 and thereby open a proximal portion 109 of the antegrade flushing fluid flow path between the stretchable membrane 106b and the catheter shaft 5.

If the membrane 106b is formed from a stretchable material, then the physician can control distension of the membrane 106b by pumping flushing fluid at a specific combination of fluid pressure and fluid flow rate, thereby controlling the size of the annular space which should be formed between the stretchable membrane 106b and the vessel wall 1a during the final step of each treatment cycle in accordance with the method of the invention. Such annular space 18, illustrated in FIGS. 15 and 16, forms a fluid flow path between the stretchable membrane 106b and the vessel wall 1a for the retrograde flow of flushing fluid across the treated stenotic area.

In a further modification of this configuration, the drainage sheath 15 may be provided with a stretchable sleeve 120 surrounding the drainage sheath 15 and having one end connected to the drainage sheath 15 close to its distal end. At or near the other end of the stretchable sleeve the potential space between the sheath and the sleeve may be connected to an inflation source so that the space between the sheath and the sleeve becomes inflated when inflation medium is supplied into such potential space. In this embodiment, the stretchable sleeve 120 which extends over the drainage sheath 15 defines an inflatable element 117 of the sheath and eliminates the need for a separate inflation lumen within the wall of the drainage sheath.

Another modified embodiment is shown in FIG. 17. In this embodiment, the catheter shaft 5 has a long stretchable membrane 206b extending in a longitudinal direction around the shaft and the dilatation balloon 206a so that the entire antegrade flushing fluid flow path 209 is formed integrally by the stretchable membrane 206b. In the embodiment shown in FIG. 17, the dilatation balloon and separate balloon inflation lumen within the catheter shaft 5 have been replaced with an integrally formed balloon inflation lumen 208 and dilatation balloon 206a, the balloon inflation lumen 208 extending in a longitudinal direction around the catheter shaft 5 so that the catheter shaft 5 need now only to have a single lumen 7 for the guidewire 3 rather than separate guidewire and balloon inflation lumens. The inflation medium can now be pumped directly into the integrally formed balloon inflation lumen 208 and dilatation balloon 206a. As described previously, the flushing fluid openings 13 are preferably provided in the transitional segment 406 of the membrane 206b extending between the maximum diameter section 306 and the minimum diameter segment 506 of the membrane 206b.

FIGS. 20 to 23 are a series of drawings to illustrate how a self-expanding stent 600 may be deployed across a stenotic lesion 2 within a blood vessel 1 using the angioplasty device of the present invention.

FIG. 20 shows the arrangement prior to deployment of the self-expanding stent 600 from the distal end 601a of a tubular advancing catheter 601. The self-expanding stent 600 is compressed around the angioplasty balloon of the invention and inserted together with the angioplasty balloon into the distal end of the tubular advancing catheter 601 prior to advancement of the tubular advancing catheter 601 across the stenotic lesion 2 within the blood vessel 1.

As shown in FIG. 20, the tubular advancing catheter 601 is positioned within the blood vessel 1 so that both the balloon angioplasty device of the invention and the self-expanding stent 600 are located across the stenotic lesion 2. Once in this position, the tubular advancing catheter 601 is withdrawn proximally, in the direction of arrow S as shown in FIG. 21, whilst the inflatable element 6 and the self-expanding stent 600 both remain stationary relative to the stenotic lesion 2. As the tubular advancing catheter 601 is withdrawn, the self-expanding stent 600 emerges from its distal end 601a and its inherent resiliency causes it to expand within the vessel 1 and to contact the stenotic lesion 2.

As shown in the drawings, the self-expanding stent 600 may be formed from a number of elongate struts 603 which, when compressed and constrained within the tubular advancing catheter 601, lie at angles (“α” in FIG. 21) relative to the longitudinal axis “T-T” of the tubular advancing catheter 601. However, once the struts 603 emerge from the distal end 601a of the tubular advancing catheter 601, they expand radially within the vessel 1 so as to contact the stenotic lesion 2 and so assume larger angles (“β” in FIG. 21) relative to the longitudinal axis “T-T” of the tubular advancing catheter 601. It will be appreciated that the angle “β” are larger than angles “α” but that the struts 603 of the self-expanding stent 600 are still partially constrained from expanding to their fullest extent by the stenotic lesion 2.

FIG. 22 shows the side sectional view of FIGS. 20 and 21 after the self-expanding stent 600 has been fully released from the restrains of the tubular advancing catheter 601 by continuing to withdraw the tubular advancing catheter 601 whilst maintaining the stationary position of the inflatable element 6 and the self-expanding stent 600 relative to the stenotic lesion 2. As can be seen from FIG. 22, all the struts 603 of the self-expanding stent 600 have partially expanded and are in contact with the stenotic lesion 2.

To maintain the position of the self-expanding stent 600 and the inflatable element 6 of the balloon angioplasty device of the invention whilst the tubular advancing catheter 601 is withdrawn, a tubular retention member 604 is disposed within the tubular advancing catheter 601, over which the tubular advancing catheter 601 slides as it is withdrawn from the blood vessel 1 following positioning of the self-expanding stent 600 across the stenotic lesion 2. Only a distal end portion of this retention member 604 is shown in FIGS. 20 to 23 but it can be seen that it has a region 605 of reduced wall thickness at is distal end to accommodate the proximal end portion of the angioplasty balloon of the invention 6. An internal shoulder 606 provides transition between the region of reduced wall thickness 605 and thicker proximal portion 607 of the tubular retention member 604 and this shoulder 606 prevents the angioplasty balloon of the invention 6 from sliding or otherwise moving proximally into the retention member 604 when the tubular advancing catheter 601 is withdrawn in a proximal direction.

Once the tubular advancing catheter 601 has been fully withdrawn, the angioplasty balloon of the invention 6 is inflated, as shown in FIG. 23, so as to dilate the stenotic lesion 2. Dilation of the stenotic lesion 2 allows the struts 603 of the self-expanding stent 600 to assume their final, fully expanded, position in which the struts 603 lie at an angle “y” relative to the longitudinal axis “T” of the tubular advancing catheter 601, as shown in FIG. 23. Once full expansion of the struts 603 has taken place, the balloon angioplasty device of the invention is operated as previously described to provide a retrograde flow of flushing fluid across the treated stenotic lesion to remove from the treated vessel any debris which may be released from the treated stenotic lesion. Once the treatment is complete, the angioplasty balloon of the invention is withdrawn from the blood vessel 1 leaving the fully expanded self-expanding stent 600 in position within the blood vessel 1 to maintain the treatment site in its dilated state.

Many modifications and variations of the invention falling within the terms of the following claims will be apparent to those skilled in the art and the foregoing description should be regarded as a description of the preferred embodiments only.

1-50. (canceled)

51. A balloon angioplasty device for dilating a region of a vessel of a patient which has been at least partially occluded by the presence of a stenotic lesion in the vessel, the device comprising an elongate catheter shaft having an inflatable element located around the catheter shaft proximal to its distal end, the inflatable element comprising a dilatation balloon and a membrane which extends in a longitudinal direction around the dilatation balloon and has at least one flushing fluid opening located at or near its distal end, at least the dilatation balloon being attached at its distal end to the catheter shaft, the balloon angioplasty device also including a balloon inflation lumen in communication with the dilatation balloon and an antegrade flushing fluid flow path, a distal portion of said flushing fluid flow path being located between the dilatation balloon and the membrane so that, when the angioplasty device has been advanced over a guidewire into the vessel to be treated and the inflatable element of the angioplasty device has been positioned across the stenotic lesion of the vessel, the dilatation balloon is inflated by supplying pressurised inflation medium through the balloon inflation lumen into the dilatation balloon to cause the inflatable element to dilate the stenotic lesion in the vessel, the inflation of the dilatation balloon being followed by its partial deflation during which the membrane continues to be pressed against the treated stenotic lesion by the pressure of flushing fluid which is pumped along the antegrade flushing fluid flow path between the dilatation balloon and the membrane, said pressurised flushing fluid entering the treated vessel through the or each flushing fluid opening(s) located at or near the distal end of the membrane, the membrane being pressed against the treated stenotic lesion to prevent the release of debris therefrom until the pressure of fluid in the vessel distal to the inflatable element increases to a pressure at which at least a portion of the flushing fluid that has entered the vessel through the or each flushing fluid opening(s) in the membrane is forced to flow in a retrograde direction between the treated stenotic lesion and the membrane when the membrane moves away from the treated stenotic lesion thereby providing at least one retrograde fluid flow path for the retrograde flowing flushing fluid which entrains debris released from the stenotic lesion for subsequent removal of the retrograde flowing flushing fluid and debris entrained in such retrograde flowing flushing fluid from the treated vessel.

52. A balloon angioplasty device according to claim 51, wherein the antegrade flushing fluid flow path includes a flushing fluid supply lumen which extends in a longitudinal direction within the catheter shaft and communicates distally with the distal portion of the antegrade flushing fluid flow path located between the dilatation balloon and the membrane of the inflatable element.

53. A balloon angioplasty device according to claim 52, wherein the flushing fluid supply lumen has at least one distal opening which is located along the catheter shaft distal to an area of a proximal attachment of the membrane to the catheter shaft and proximal to an inflatable portion of the dilatation balloon.

54. A balloon angioplasty device according to claim 53, wherein the or each distal opening(s) of the flushing fluid supply lumen is located along the catheter shaft proximal to a proximal end of the dilatation balloon.

55. A balloon angioplasty device according to claim 51, wherein the membrane of the inflatable element, when inflated by the dilatation balloon, has a maximum diameter section which extends in a longitudinal direction around a maximum diameter section of the dilatation balloon, the maximum diameter section of the membrane continues distally into a transitional segment of the membrane which becomes progressively smaller in diameter as it converges towards the catheter shaft and finally continues into a minimum diameter segment of the membrane which is connected to the catheter shaft, the transitional segment of the membrane including the or each flushing fluid opening(s) of the membrane.

56. A balloon angioplasty device of claim 55, wherein the maximum diameter section of the membrane both distally and proximally extends beyond corresponding distal and proximal ends of the maximum diameter section of the dilatation balloon.

57. A balloon angioplasty device according to claim 51, wherein both dilatation balloon and the membrane are designed to be able together to withstand a certain maximum predetermined pressure of the inflation medium within the dilatation balloon and wherein at least the dilatation balloon is designed to rupture when the pressure within the balloon exceeds a certain predetermined burst pressure.

58. A balloon angioplasty device according to claim 51, wherein both the dilatation balloon and the membrane are made from substantially non-stretchable material, the substantially non-stretchable membrane and the dilatation balloon being attached to each other at locations spaced around the balloon so that the membrane moves away from the treated stenotic lesion by being pulled away from the treated stenotic lesion by the collapsing dilatation balloon in the region extending over at least one location of attachment between the dilatation balloon and the membrane.

59. A balloon angioplasty device according to claim 58, wherein the dilatation balloon and the substantially non-stretchable membrane are attached to each other at said spaced locations around the balloon along at least two longitudinally extending join lines or strips.

60. A method of using a balloon angioplasty device for dilating a region of a vessel of a patient which has been at least partially occluded by the presence of a stenotic lesion in the vessel, the device comprising an elongate catheter shaft having an inflatable element located around the catheter shaft proximal to its distal end, the inflatable element comprising a dilatation balloon and a membrane which extends in a longitudinal direction around the dilatation balloon and has at least one flushing fluid opening located at or near its distal end, at least the dilatation balloon being attached at its distal end to the catheter shaft, the balloon angioplasty device also including a balloon inflation lumen in communication with the dilatation balloon and an antegrade flushing fluid flow path, a distal portion of said flushing fluid flow path being located between the dilatation balloon and the membrane, the method comprising the steps of:
(a) positioning the inflatable element of the angioplasty device across the stenotic lesion by advancing the angioplasty device over a guidewire into a vessel to be treated, the guidewire being placed in the vessel prior to being received in a guidewire lumen of the angioplasty device;
(b) inflating the dilatation balloon by pumping inflation medium through the balloon inflation lumen into the dilatation balloon to cause the inflatable element to dilate the vessel;
(c) partially deflating the dilatation balloon and maintaining the membrane pressed against the treated stenotic lesion to prevent release of debris therefrom by pumping flushing fluid through the antegrade flushing fluid flow path so that it flows through the distal portion of said flow path located between the dilatation balloon and the membrane and enters the treated vessel through the or each flushing fluid opening(s) in the membrane located at or near the distal end of the membrane;
(d) further deflating the dilatation balloon so that the membrane moves away from the treated stenotic lesion to form a retrograde fluid flow path between the treated stenotic lesion and the membrane for the retrograde flow of at least a portion of the flushing fluid that has entered the vessel through the or each flushing fluid opening(s) in the membrane to entrain debris released from the stenotic lesion in the retrograde flow of flushing fluid for subsequent removal of said flushing fluid and debris entrained in the flushing fluid from the treated vessel.
